(19) United States
(12) Patent Application Publication
(10) Pub. No.: US 2023/0303542 A1
(43) Pub. Date: Sep. 28, 2023

(54) SOLID FORMS OF A PARP14 INHIBITOR

(71) Applicant: Ribon Therapeutics, Inc., Cambridge, MA (US)

(72) Inventors: Melissa Marie Vasbinder, Newton, MA (US); Laurie B. Schenkel, Somerville, MA (US); Kerren Kalai Swinger, Lexington, MA (US); Kevin Wayne Kuntz, Woburn, MA (US); Nicholas Robert Perl, Somerville, MA (US); John Alphons Kremers, Cynwyd, Corwen (GB); Zheng Jane Li, Kendall Park, NJ (US); Pengyuan Chen, Beijing (CN)

(21) Appl. No.: 18/107,164

(22) Filed: Feb. 8, 2023

(30) Foreign Application Priority Data
Feb. 9, 2022 (WO) PCT/CN2022/075605

(51) Int. Cl. C07D 405/14

(52) U.S. Cl. CPC C07D 405/14; C07B 2200/13

(57) ABSTRACT

The present invention relates to solid forms of the poly(ADP-ribose) polymerase 14 (PARP14) inhibitor 7-((1-acetylpiperidin-4-yl)methoxy)-5-fluoro-2-(((tetrahydro-2H-pyran-4-yl)thio)methyl)quinazolin-4(3H)-one, including methods of preparation thereof, where the inhibitor is useful in the treatment of cancer and inflammatory diseases.

### FIELD OF THE INVENTION

The present invention relates to solid forms of the poly(ADP-ribose) polymerase 14 (PARP14) inhibitor 7-((1-acetylpiperidin-4-yl)methoxy)-5-fluoro-2-(((tetrahydro-2H-pyran-4-yl)thio)methyl)quinazolin-4(3H)-one, including methods of preparation thereof, where the inhibitor is useful in the treatment of cancer and inflammatory diseases.

### BACKGROUND OF THE INVENTION

Poly(ADP-ribose) polymerases (PARPs) are members of a family of seventeen enzymes that regulate fundamental cellular processes including gene expression, protein degradation, and multiple cellular stress responses (Vyas S, et al. Nat Rev Cancer. 2014 Jun 5;14(7):502-509). The ability of cancer cells to survive under stress is a fundamental cancer mechanism and an emerging approach for novel therapeutics. One member of the PARP family, PARP1, has already been shown to be an effective cancer target in connection to cellular stress induced by DNA damage, either induced by genetic mutation or with cytotoxic chemotherapy, with four approved drugs in the clinic and several others in late stage development (Ohmoto A, et al. OncoTargets and Therapy. 2017;Volume 10:5195).

The seventeen members of the PARP family were identified in the human genome based on the homology within their catalytic domains (Vyas S, et al. Nat Commun. 2013 Aug 7;4:2240). However, their catalytic activities fall into 3 different categories. The majority of PARP family members catalyze the transfer of mono- ADP-ribose units onto their substrates (monoPARPs), while others (PARP1, PARP2, TNKS, TNKS2) catalyze the transfer of poly-ADP-ribose units onto substrates (polyPARPs). Finally, PARP13 is thus far the only PARP for which catalytic activity could not be demonstrated either in vitro or in vivo. PARP14 is a cytosolic as well as nuclear monoPARP. It was originally identified as BAL2 (B Aggressive Lymphoma 2), a gene associated with inferior outcome of diffuse large B cell lymphoma (DLBCL), together with two other monoPARPs (PARP9 or BAL1 and PARP15 or BAL3) (Aguiar RC, et al. Blood. 2000 Dec 9;96(13):4328-4334 and Juszczynski P, et al. Mol Cell Biol. 2006 Jul 1;26(14):5348-5359). PARP14, PARP9 and PARP15 are also referred to as macro-PARPs due to the presence of macro-domains in their N-terminus. The genes for the three macroPARPs are located in the same genomic locus suggesting co-regulation. Indeed, the gene expression of PARP14 and PARP9 is highly correlated across normal tissues and cancer types. PARP14 is overexpressed in tumors compared to normal tissues, including established cancer cell lines in comparison to their normal counterparts. Literature examples of cancers with high PARP14 expression are DLBCL (Aguiar RCT, et al. J Biol Chem. 2005 Aug 1;280(40):33756-33765), multiple myeloma (MM) (Barbarulo A, et al. Oncogene. 2012 Oct 8;32(36):4231-4242) and hepatocellular carcinoma (HCC) (Iansante V, et al. Nat Commun. 2015 Aug 10;6:7882). In MM and HCC cell lines RNA interference (RNAi) mediated PARP14 knockdown inhibits cell proliferation and survival. Other studies show that the enzymatic activity of PARP14 is required for survival of prostate cancer cell lines in vitro (Bachmann SB, et al. Mol Cancer. 2014 May 27;13:125).

PARP14 has been identified as a downstream regulator of IFN-γ and IL-4 signaling, influencing transcription downstream of STAT1 (in the case of IFN-γ) (Iwata H, et al. Nat Commun. 2016 Oct 31;7:12849) or STAT6 (in the case of IL-4) (Goenka S, et al. Proc Natl Acad Sci USA. 2006 Mar 6;103(11):4210-4215; Goenka S, et al. J Biol Chem. 2007 May 3;282(26):18732-18739; and Mehrotra P, et al. J Biol Chem. 2010 Nov 16;286(3):1767-1776). Parp14 -/- knockout (KO) mice have reduced marginal zone B cells, and the ability of IL-4 to confer B cell survival in vitro was reduced as well in the Parp14 KO setting (Cho SH, et al. Blood. 2009 Jan 15;113(11):2416-2425). This decreased survival signaling was linked mechanistically to decreased abilities of Parp14 KO B cells to sustain metabolic fitness and to increased Mcl-1 expression. Parp14 KO can extend survival in the Eµ-Myc lymphoma model, suggesting a role of PARP14 in Myc-driven lymphomagenesis (Cho SH, et al. Proc Natl Acad Sci USA. 2011 Sep 12;108(38):15972-15977). Gene expression data point towards roles of PARP14 in human B cell lymphoma as well. The BAL proteins, including PARP14, are highly expressed in host response (HR) DLBCLs, a genomically defined B cell lymphoma subtype characterized with a brisk inflammatory infiltrate of T and dendritic cells and presence of an IFN-y gene signature (Molecular profiling of diffuse large B-cell lymphoma identifies robust subtypes including one characterized by host inflammatory response. Monti S, et al. Blood. 2005;105(5):1851). Indeed, PARP14 is believed to be an interferon stimulated gene with its mRNA increased by stimulation of various cell systems with all types of interferon (I, II and III; www.interferome.org).

Due to its role downstream of IL-4 and IFN-γ signaling pathways PARP14 has been implicated in T helper cell and macrophage differentiation. Genetic PARP14 inactivation in macrophages skews to a pro-inflammatory M1 phenotype associated with antitumor immunity while reducing a pro-tumor M2 phenotype. M1 gene expression, downstream of IFN-γ, was found to be increased while M2 gene expression, downstream of IL-4, was decreased with PARP14 knockout or knockdown in human and mouse macrophage models. Similarly, genetic PARP14 knockout has been shown to reduce a Th2 T helper cell phenotype in the setting of skin and airway inflammation, again pertaining to the regulatory role of PARP14 in IL-4 signal transduction (Mehrotra P, et al. J Allergy Clin Immunol. 2012 Jul 25;131(2):521 and Krishnamurthy P, et al. Immunology. 2017 Jul 27;152(3):451-461).

PARP14 was shown to regulate the transcription of STAT6 (activator of transcription 6) and promotes T_{H}2 responses in T cells and B cells, which are known to promote allergic airway disease (asthmatic condition). Genetic depletion of PARP14 and its enzymatic activity in a model of allergic airway disease led to reduced lung inflammation and IgE levels, which are key readouts of the asthmatic process in this model. In addition, the enzymatic activity of PARP14 promoted a T_{H}2 phenotype differentiation in a STAT6 dependent manner. (Mehrotra P, et al. J Allergy Clin Immunol. 2012 Jul 25;131(2):521) Therefore, inhibition of the PARP14 catalytic activity may be a potential novel therapy for allergic airway disease.

Accordingly, there is a need for new solid forms of PARP14-inhibiting molecules for preparing pharmaceutically useful formulations and dosage forms with suitable properties related to, for example, facilitating the manufacture of safe, effective, and high quality drug products.

### SUMMARY OF THE INVENTION

The present invention is directed to solid forms of 7-((1-acetylpiperidin-4-yl)methoxy)-5-fluoro-2-(((tetrahydro-2H-pyran-4-yl)thio)methyl)quinazolin-4(3H)-one.

The present invention is further directed to crystalline forms of the solid forms described herein.

The present invention is further directed to pharmaceutical compositions comprising a solid form described herein, and at least one pharmaceutically acceptable carrier.

The present invention is further directed to therapeutic methods of using the solid forms described herein. The present disclosure also provides uses of the solid forms described herein in the manufacture of a medicament for use in therapy. The present disclosure also provides the solid forms described herein for use in therapy.

The present invention is further directed to processes for preparing the solid forms described herein.

### BRIEF DESCRIPTION OF THE DRAWINGS

FIG. 1 shows the X-ray powder diffraction (XRPD) pattern of Form I.

FIG. 2 shows the differential scanning calorimetry (DSC) and thermogravimetric analysis (TGA) thermogram of Form I.

FIG. 3 shows the polarized light microscopy (PLM) micrograph of Form I.

FIG. 4 shows the XRPD pattern of Form II.

FIG. 5 shows the DSC and TGA thermogram of Form II.

FIG. 6 shows the PLM micrograph of Form II.

FIG. 7 shows the dynamic vapor sorption (DVS) plot of Form II.

FIG. 8 shows the XRPD pattern of Form III.

FIG. 9 shows the XRPD pattern of Form IV.

FIG. 10 shows the DSC and TGA thermogram of Form IV.

FIG. 11 shows the XRPD pattern of Form V.

FIG. 12 shows the DSC and TGA thermogram of Form V.

FIG. 13 shows the XRPD pattern of Form VI.

FIG. 14 shows the DSC and TGA thermogram of Form VI.

### DETAILED DESCRIPTION

The present invention is directed to, inter alia, solid forms of 7-((1-acetylpiperidin-4-yl)methoxy)-5-fluoro-2-(((tetrahydro-2H-pyran-4-yl)thio)methyl)quinazolin-4(3H)-one (Compound 1), the structure of which is shown below.

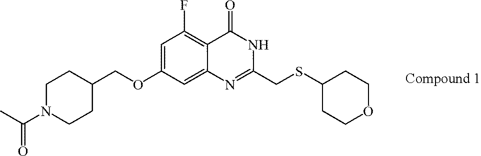 [SMILES: CC(=O)N1CCC(COc2cc(F)c3c(=O)[nH]c(CSC4CCOCC4)nc3c2)CC1]

The preparation of Compound 1 is described in U.S. Pat. No. 10,562,891, which is incorporated by reference in its entirety.

Compound 1 and its salts can be isolated as one or more solid forms. The solid forms (including, e.g., crystalline forms) described herein have many advantages, for example they have desirable properties, such as ease of handling, ease of processing, storage stability, and ease of purification. Moreover, the crystalline forms can be useful for improving the performance characteristics of a pharmaceutical product such as dissolution profile, shelf-life and bioavailability.

Compound 1 can be prepared in various crystalline forms including, e.g., Form I, Form II, Form III, Form IV, Form V, or Form VI. In some embodiments, the solid form of Compound 1 is amorphous.

As used herein, the phrase “solid form” refers to a compound provided herein in either an amorphous state or a crystalline state (“crystalline form” or “crystalline solid” or “crystalline solid form”), whereby a compound provided herein in a crystalline state may optionally include solvent or water within the crystalline lattice, for example, to form a solvated or hydrated crystalline form. In some embodiments, the compound provided herein is in a crystalline state as described herein.

As used herein, the term “peak” or “characteristic peak” refers to an XRPD reflection having a relative height/intensity of at least about 3% of the maximum peak height/intensity.

As used herein, the term “crystalline” or “crystalline form” refers to a crystalline solid form of a chemical compound, including, but not limited to, a single-component or multiple-component crystal form, e.g., including solvates, hydrates, clathrates, and a co-crystal. For example, crystalline means having a regularly repeating and/or ordered arrangement of molecules, and possessing a distinguishable crystal lattice. The term “crystalline form” is meant to refer to a certain lattice configuration of a crystalline substance. Different crystalline forms of the same substance typically have different crystalline lattices (e.g., unit cells), typically have different physical properties attributed to their different crystalline lattices, and in some instances, have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the crystalline form as well as help determine stability and solvent/water content.

Different crystalline forms of a particular substance, such as Compound 1 as described herein, can include both anhydrous forms of that substance and solvated/hydrated forms of that substance, where each of the anhydrous forms and solvated/hydrated forms are distinguished from each other by different XRPD patterns, or other solid state characterization methods, thereby signifying different crystalline lattices. In some instances, a single crystalline form (e.g., identified by a unique XRPD pattern) can have variable water or solvent content, where the lattice remains substantially unchanged (as does the XRPD pattern) despite the compositional variation with respect to water and/or solvent.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, filters used, the sample mounting procedure, and the particular instrument employed. In some instances, new peaks may be observed or existing peaks may disappear, depending on the type of the machine or the settings (for example, whether a Ni filter is used or not). As used herein, the term “peak” refers to a reflection having a relative height/intensity of at least about 3% or at least about 4% of the maximum peak height/intensity. Moreover, instrument variation and other factors can affect the 2-theta values. Thus, peak assignments, such as those reported herein, can vary by plus or minus about 0.2° (2-theta) and the term “substantially” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc. Accordingly, a crystalline form reported herein having a DSC thermogram “substantially” as shown in any of the Figures is understood to accommodate such variation.

Crystalline forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, exposure to moisture, grinding and solvent-drop grinding.

As used herein, the term “amorphous” or “amorphous form” is intended to mean that the substance, component, or product in question is not crystalline as determined, for instance, by XRPD or where the substance, component, or product in question, for example is not birefringent when viewed microscopically. For example, amorphous means essentially without regularly repeating arrangement of molecules or lacks the long range order of a crystal, i.e., amorphous form is non-crystalline. An amorphous form does not display a defined x-ray diffraction pattern with sharp maxima. In certain embodiments, a sample comprising an amorphous form of a substance may be substantially free of other amorphous forms and/or crystalline forms. For example, an amorphous substance can be identified by an XRPD spectrum having an absence of reflections.

Compound 1 can be prepared in batches referred to as batches, samples, or preparations. The batches, samples, or preparations can include Compound 1 in any of the crystalline or non-crystalline forms described herein, including hydrated and non-hydrated forms, and mixtures thereof.

Compounds provided herein (e.g., Compound 1) can also include all isotopes of atoms occurring in the intermediates or final compounds. Isotopes include those atoms having the same atomic number but different mass numbers. For example, isotopes of hydrogen include tritium and deuterium. One or more constituent atoms of the compounds provided herein can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7 or 8 deuterium atoms. Synthetic methods for including isotopes into organic compounds are known in the art.

In some embodiments, Compound 1 is substantially isolated. The term “substantially isolated” is meant that the compound is at least partially or substantially separated from the environment in which it was formed or detected. Partial separation can include, e.g., a composition enriched in the compound, salts, hydrates, solvates, or solid forms provided herein. Substantial separation can include compositions containing at least about 50%, at least about 60%, at least about 70%, at least about 80%, at least about 90%, at least about 95%, at least about 97%, or at least about 99% by weight of the compound, salts, hydrates, solvates, or solid forms provided herein.

The term “hydrate,” as used herein, is meant to refer to a solid form of Compound 1 that includes water. The water in a hydrate can be present in a stoichiometric amount with respect to the amount of salt in the solid, or can be present in varying amounts, such as can be found in connection with channel hydrates.

As used herein, the term “substantially” when referring to a characteristic figure of a crystal form, such as an XRPD pattern, a DSC thermogram, a TGA thermogram, or the like, means that a subject figure may be non-identical to the reference depicted herein, but it falls within the limits of experimental error and thus may be deemed as derived from the same crystal form as disclosed herein, as judged by a person of ordinary skill in the art.

As used herein, the term “substantially crystalline,” means a majority of the weight of a sample or preparation of Compound 1 is crystalline and the remainder of the sample is a non-crystalline form (e.g., amorphous form) of the same compound. In some embodiments, a substantially crystalline sample has at least about 95% crystallinity (e.g., about 5% of the non-crystalline form of the same compound), at least about 96% crystallinity (e.g., about 4% of the non-crystalline form of the same compound), at least about 97% crystallinity (e.g., about 3% of the non-crystalline form of the same compound), at least about 98% crystallinity (e.g., about 2% of the non-crystalline form of the same compound), at least about 99% crystallinity (e.g., about 1% of the non-crystalline form of the same compound), or about 100% crystallinity (e.g., about 0% of the non-crystalline form of the same compound). In some embodiments, the term “fully crystalline” means at least about 99% or about 100% crystallinity.

As used herein, the term “% crystallinity” or “crystalline purity,” means percentage of a crystalline form in a preparation or sample which may contain other forms such as an amorphous form of the same compound, or at least one other crystalline form of the compound, or mixtures thereof. In some embodiments, the crystalline forms can be isolated with a purity of at least about 80%, about 85%, about 90%, about 95%, about 96%, about 97%, about 98%, or about 99%. In some embodiments, the crystalline forms can be isolated with a purity greater than about 99%.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “melting point” refers to an endothermic event or endothermal event observed in e.g., a DSC experiment. An endothermic event is a process or reaction in which a sample absorbs energy from its surrounding in the form of e.g., heat as in a DSC experiment. An exothermic event is a process or reaction in which a sample releases energy. The process of heat absorption and release can be detected by DSC. In some embodiments, the term “melting point” is used to describe the major endothermic event revealed on a particular DSC thermogram.

The term “room temperature” or “RT” as used herein, is understood in the art, and refers generally to a temperature, e.g., a reaction temperature, that is about the temperature of the room in which the reaction is carried out, for example, a temperature from about 20° C. to about 30° C.

The term “elevated temperature” as used herein, is understood in the art, and refer generally to a temperature, e.g., a reaction temperature, that is above room temperature, e.g., above 30° C.

### Compound 1 Form I

In some embodiments, Compound 1 is crystalline and has the characteristics of Form I described below. The data characterizing Form I are consistent with a hydrate.

In some embodiments, Form I has characteristic XRPD peaks at about 6.3, about 8.9, about 16.9, and about 22.4 degrees 2-theta. In some embodiments, Form I has at least one characteristic XRPD peak selected from about 6.3, about 8.9, about 16.9, and about 22.4 degrees 2-theta. In some embodiments, Form I has at least two characteristic XRPD peaks selected from about 6.3, about 8.9, about 16.9, and about 22.4 degrees 2-theta. In some embodiments, Form I has at least three characteristic XRPD peaks selected from about 6.3, about 8.9, about 16.9, and about 22.4 degrees 2-theta. In some embodiments, Form I has a characteristic XRPD peak at about 6.3 degrees 2-theta. In some embodiments, Form I has a characteristic XRPD peak at about 8.9 degrees 2-theta. In some embodiments, Form I has a characteristic XRPD peak at about 16.9 degrees 2-theta. In some embodiments, Form I has a characteristic XRPD peak at about 22.4 degrees 2-theta.

In some embodiments, Form I has at least one characteristic XRPD peak selected from about 6.3, about 8.9, about 12.5, about 14.3, about 15.3, about 16.9, about 17.7, about 19.0, about 22.4, and about 23.8 degrees 2-theta. In some embodiments, Form I has at least two characteristic XRPD peaks selected from about 6.3, about 8.9, about 12.5, about 14.3, about 15.3, about 16.9, about 17.7, about 19.0, about 22.4, and about 23.8 degrees 2-theta. In some embodiments, Form I has at least three characteristic XRPD peaks selected from about 6.3, about 8.9, about 12.5, about 14.3, about 15.3, about 16.9, about 17.7, about 19.0, about 22.4, and about 23.8 degrees 2-theta. In some embodiments, Form I has at least four characteristic XRPD peaks selected from about 6.3, about 8.9, about 12.5, about 14.3, about 15.3, about 16.9, about 17.7, about 19.0, about 22.4, and about 23.8 degrees 2-theta. In some embodiments, Form I has at least five characteristic XRPD peaks selected from about 6.3, about 8.9, about 12.5, about 14.3, about 15.3, about 16.9, about 17.7, about 19.0, about 22.4, and about 23.8 degrees 2-theta. In some embodiments, Form I has characteristic XRPD peaks at about 6.3, about 8.9, about 12.5, about 14.3, about 15.3, about 16.9, about 17.7, about 19.0, about 22.4, and about 23.8 degrees 2-theta.

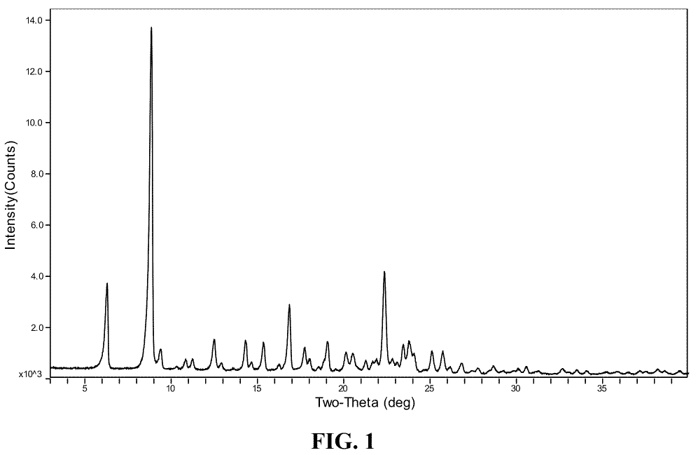

In some embodiments, Form I has an XRPD pattern with characteristic peaks as substantially shown in FIG. 1.

In some embodiments, Form I has a DSC thermogram comprising an endotherm onset at a temperature of about 82° C. In some embodiments, Form I has a DSC thermogram comprising an endotherm peak at a temperature of about 92° C. In some embodiments, Form I has a DSC thermogram comprising an endotherm onset at a temperature of about 82° C. and an endotherm peak at a temperature of about 92° C. In some embodiments, Form I has a DSC thermogram comprising an endotherm onset at a temperature of about 189° C. In some embodiments, Form I has a DSC thermogram comprising an endotherm peak at a temperature of about 192° C. In some embodiments, Form I has a DSC thermogram comprising an endotherm onset at a temperature of about 189° C. and an endotherm peak at a temperature of about 192° C.

In some embodiments, Form I shows a weight loss of about 1.1% when heated to about 110° C.

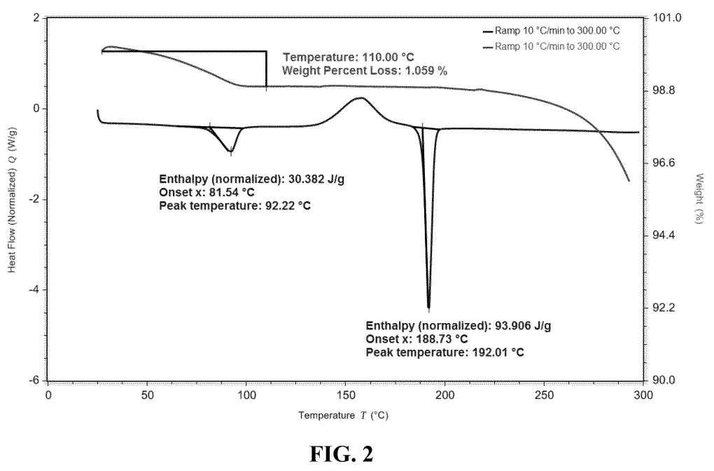

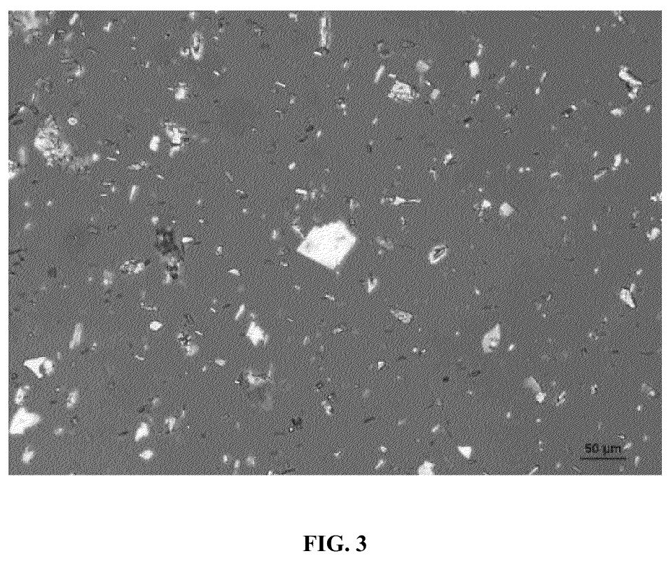

In some embodiments, Form I has a DSC thermogram substantially as depicted in FIG. 2. In some embodiments, Form I has a TGA thermogram substantially as depicted in FIG. 2. In some embodiments, Form I has a PLM substantially as depicted in FIG. 3.

In some embodiments, Form I can be isolated with a crystalline purity of at least about 80%, about 85%, about 90%, about 95%, about 96%, about 97%, about 98%, or about 99%. In some embodiments, Form I can be isolated with a crystalline purity greater than about 99%. In some embodiments, Form I can be isolated with a crystalline purity greater than about 99.9%. In some embodiments, Form I is substantially free of other crystalline form. In some embodiments, Form I is substantially free of amorphous form.

In some embodiments, Form I is prepared by precipitating Compound 1 from a solution comprising Compound 1 and S1, wherein S1 is a solvent. In some embodiments, the solution of Compound 1 and S1 is first heated to an elevated temperature before the precipitating. In some embodiments, the elevated temperature is between 60° C. and 100° C. In some embodiments, the elevated temperature is between 70° C. and 90° C. In some embodiments, the elevated temperature is about 80° C. In some embodiments, the precipitating is carried out by concentration of the solution, evaporation of S1, reduction of temperature of the solution, addition of anti-solvent, or combination thereof. In some embodiments, S1 is an organic solvent. In some embodiments, S1 is an alcohol. In some embodiments, S1 is methanol.

### Compound 1 Form II

In some embodiments, Compound 1 is crystalline and has the characteristics of Form II described below. The data characterizing Form II are consistent with an anhydrous solid form.

In some embodiments, Form II has characteristic XRPD peaks at about 8.6, about 9.4, about 19.5, and about 22.7 degrees 2-theta. In some embodiments, Form II has at least one characteristic XRPD peak selected from about 8.6, about 9.4, about 19.5, and about 22.7 degrees 2-theta. In some embodiments, Form II has at least two characteristic XRPD peaks selected from about 8.6, about 9.4, about 19.5, and about 22.7 degrees 2-theta. In some embodiments, Form II has at least three characteristic XRPD peaks selected from about 8.6, about 9.4, about 19.5, and about 22.7 degrees 2-theta. In some embodiments, Form II has a characteristic XRPD peak at about 8.6 degrees 2-theta. In some embodiments, Form II has a characteristic XRPD peak at about 9.4 degrees 2-theta. In some embodiments, Form II has a characteristic XRPD peak at about 19.5 degrees 2-theta. In some embodiments, Form II has a characteristic XRPD peak at about 22.7 degrees 2-theta.

In some embodiments, Form II has at least one characteristic XRPD peak selected from about 8.6, about 9.4, about 12.2, about 14.8, about 15.9, about 16.1, about 17.7, about 18.1, about 19.5, about 20.0, about 22.7, and about 24.6 degrees 2-theta. In some embodiments, Form II has at least two characteristic XRPD peaks selected from about 8.6, about 9.4, about 12.2, about 14.8, about 15.9, about 16.1, about 17.7, about 18.1, about 19.5, about 20.0, about 22.7, and about 24.6 degrees 2-theta. In some embodiments, Form II has at least three characteristic XRPD peaks selected about 8.6, about 9.4, about 12.2, about 14.8, about 15.9, about 16.1, about 17.7, about 18.1, about 19.5, about 20.0, about 22.7, and about 24.6 degrees 2-theta. In some embodiments, Form II has at least four characteristic XRPD peaks selected from about 8.6, about 9.4, about 12.2, about 14.8, about 15.9, about 16.1, about 17.7, about 18.1, about 19.5, about 20.0, about 22.7, and about 24.6 degrees 2-theta. In some embodiments, Form II has at least five characteristic XRPD peaks selected from about 8.6, about 9.4, about 12.2, about 14.8, about 15.9, about 16.1, about 17.7, about 18.1, about 19.5, about 20.0, about 22.7, and about 24.6 degrees 2-theta. In some embodiments, Form II has characteristic XRPD peaks at about 8.6, about 9.4, about 12.2, about 14.8, about 15.9, about 16.1, about 17.7, about 18.1, about 19.5, about 20.0, about 22.7, and about 24.6 degrees 2-theta.

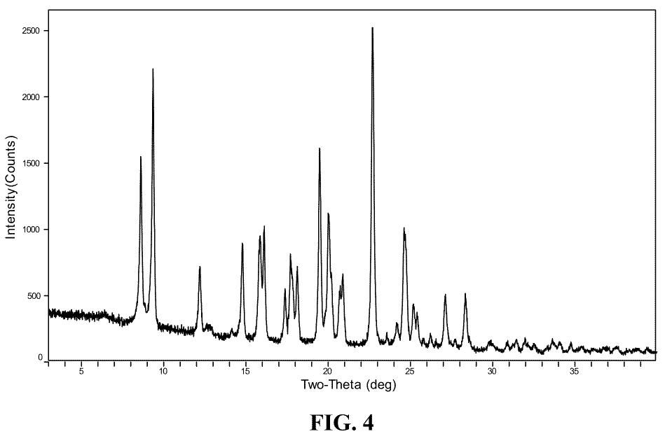

In some embodiments, Form II has an XRPD pattern with characteristic peaks as substantially shown in FIG. 4.

In some embodiments, Form II has a DSC thermogram comprising an endotherm onset at a temperature of about 189° C. In some embodiments, Form II has a DSC thermogram comprising an endotherm peak at a temperature of about 190° C. In some embodiments, Form II has a DSC thermogram comprising an endotherm onset at a temperature of about 189° C. and an endotherm peak at a temperature of about 190° C.

In some embodiments, Form II shows a weight loss less than 0.1% when heated to about 150° C. In some embodiments, Form II shows no weight loss when heated to about 150° C.

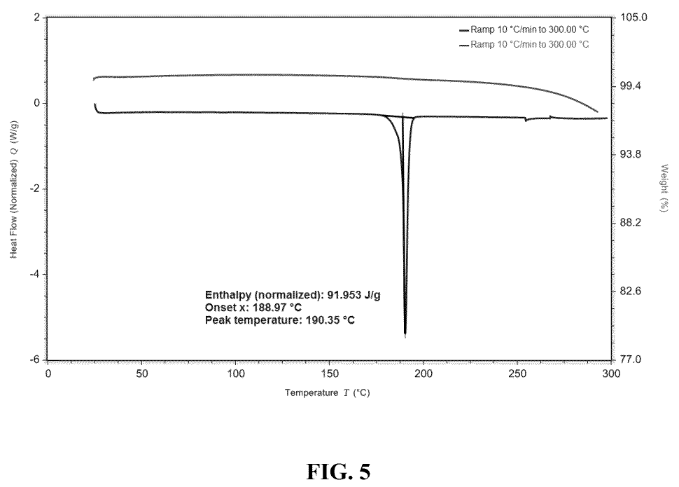

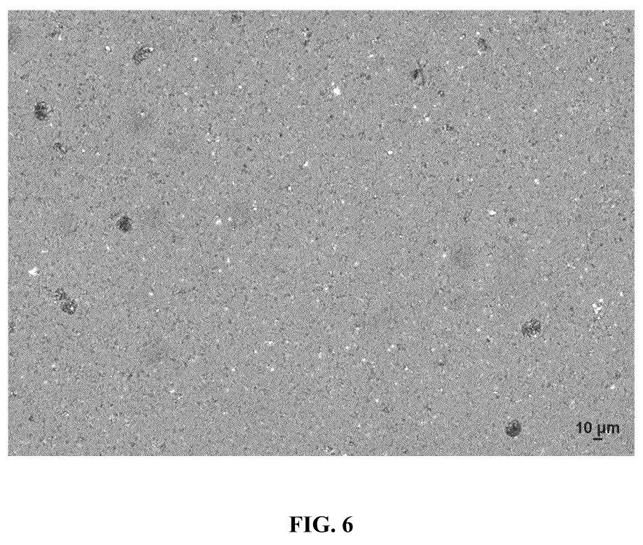

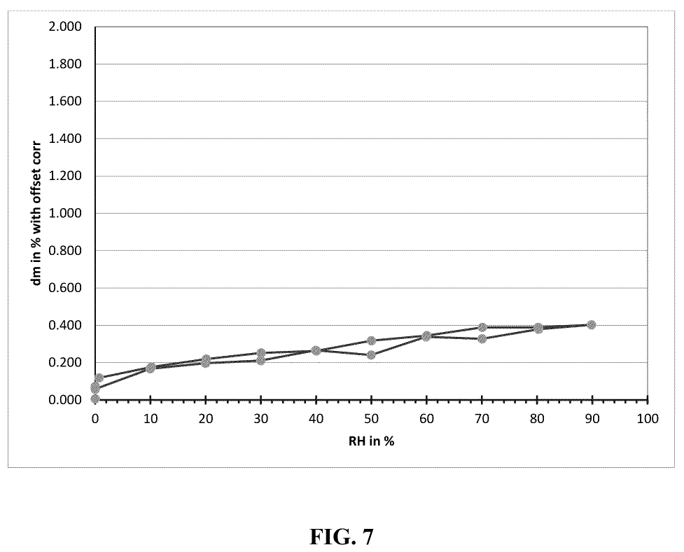

In some embodiments, Form II has a DSC thermogram substantially as depicted in FIG. 5. In some embodiments, Form II has a TGA thermogram substantially as depicted in FIG. 5. In some embodiments, Form II has a PLM substantially as depicted in FIG. 6. In some embodiments, Form II has a DVS plot substantially as depicted in FIG. 7.

In some embodiments, Form II can be isolated with a crystalline purity of at least about 80%, about 85%, about 90%, about 95%, about 96%, about 97%, about 98%, or about 99%. In some embodiments, Form II can be isolated with a crystalline purity greater than about 99%. In some embodiments, Form II can be isolated with a crystalline purity greater than about 99.9%. In some embodiments, Form II is substantially free of other crystalline form. In some embodiments, Form II is substantially free of amorphous form.

In some embodiments, Form II is prepared by precipitating Compound 1 from a solution comprising Compound 1 and S2, wherein S2 is a solvent. In some embodiments, the precipitating is carried out by concentration of the solution, evaporation of S2, reduction of temperature of the solution, addition of anti-solvent, or combination thereof. In some embodiments, S2 is an organic solvent. In some embodiments, S2 is selected from one of the following solvents: methyl ethyl ketone, MTBE, isopropyl acetate, 2-Me-THF, toluene, ethanol, ethyl acetate, THF, heptane, methanol, anisole, and mixtures thereof. In some embodiments, the precipitating is carried out by the addition of an anti-solvent. In some embodiments, the anti-solvent is heptane. In some embodiments, S2 is a mixture of methanol, anisole, and MTBE. In some embodiments, S2 is a mixture of methanol, water, and MTBE.

### Compound 1 Form III

In some embodiments, Compound 1 is crystalline and has the characteristics of Form III described below. The data characterizing Form III is consistent with a hydrate.

In some embodiments, Form III has characteristic XRPD peaks at about 6.2, about 8.9, about 16.9, about 19.0, and about 21.9 degrees 2-theta. In some embodiments, Form III has at least one characteristic XRPD peak selected from about 6.2, about 8.9, about 16.9, about 19.0, and about 21.9 degrees 2-theta. In some embodiments, Form III has at least two characteristic XRPD peaks selected from about 6.2, about 8.9, about 16.9, about 19.0, and about 21.9 degrees 2-theta. In some embodiments, Form III has at least three characteristic XRPD peaks selected from about 6.2, about 8.9, about 16.9, about 19.0, and about 21.9 degrees 2-theta. In some embodiments, Form III has at least four characteristic XRPD peaks selected from about 6.2, about 8.9, about 16.9, about 19.0, and about 21.9 degrees 2-theta. In some embodiments, Form III has a characteristic XRPD peak at about 6.2 degrees 2-theta. In some embodiments, Form III has a characteristic XRPD peak at about 8.9 degrees 2-theta. In some embodiments, Form III has a characteristic XRPD peak at about 16.9 degrees 2-theta. In some embodiments, Form III has a characteristic XRPD peak at about 19.0 degrees 2-theta. In some embodiments, Form III has a characteristic XRPD peak at about 21.9 degrees 2-theta.

In some embodiments, Form III has at least one characteristic XRPD peak selected from about 6.2, about 8.9, about 12.3, about 14.2, about 15.5, about 16.9, about 18.1, about 19.0, about 21.5, about 21.9, and about 23.8 degrees 2-theta. In some embodiments, Form III has at least two characteristic XRPD peaks selected from about 6.2, about 8.9, about 12.3, about 14.2, about 15.5, about 16.9, about 18.1, about 19.0, about 21.5, about 21.9, and about 23.8 degrees 2-theta. In some embodiments, Form III has at least three characteristic XRPD peaks selected from about 6.2, about 8.9, about 12.3, about 14.2, about 15.5, about 16.9, about 18.1, about 19.0, about 21.5, about 21.9, and about 23.8 degrees 2-theta. In some embodiments, Form III has at least four characteristic XRPD peaks selected from about 6.2, about 8.9, about 12.3, about 14.2, about 15.5, about 16.9, about 18.1, about 19.0, about 21.5, about 21.9, and about 23.8 degrees 2-theta. In some embodiments, Form III has at least five characteristic XRPD peaks selected from about 6.2, about 8.9, about 12.3, about 14.2, about 15.5, about 16.9, about 18.1, about 19.0, about 21.5, about 21.9, and about 23.8 degrees 2-theta. In some embodiments, Form III has characteristic XRPD peaks at about 6.2, about 8.9, about 12.3, about 14.2, about 15.5, about 16.9, about 18.1, about 19.0, about 21.5, about 21.9, and about 23.8 degrees 2-theta.

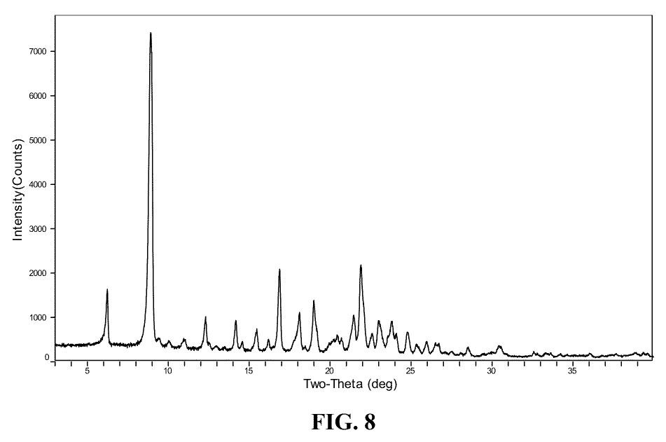

In some embodiments, Form III has an XRPD pattern with characteristic peaks as substantially shown in FIG. 8.

In some embodiments, Form III can be isolated with a crystalline purity of at least about 80%, about 85%, about 90%, about 95%, about 96%, about 97%, about 98%, or about 99%. In some embodiments, Form III can be isolated with a crystalline purity greater than about 99%. In some embodiments, Form III can be isolated with a crystalline purity greater than about 99.9%. In some embodiments, Form III is substantially free of other crystalline form. In some embodiments, Form III is substantially free of amorphous form.

In some embodiments, Form III is prepared by precipitating Compound 1 from a solution comprising Compound 1 and S3, wherein S3 is a solvent. In some embodiments, the precipitating is carried out by concentration of the solution, evaporation of S3, reduction of temperature of the solution, addition of anti-solvent, or combination thereof. In some embodiments, S3 is an organic solvent. In some embodiments, S3 is selected from one of the following solvents: acetonitrile, dichloromethane, methanol, water and mixtures thereof. In some embodiments, S3 is a mixture of methanol and acetone. In some embodiments, S3 is a mixture of acetonitrile and water. In some embodiments, the precipitating is carried out by the addition of an anti-solvent. In some embodiments, the anti-solvent is heptane.

### Compound 1 Form IV

In some embodiments, Compound 1 is crystalline and has the characteristics of Form IV described below. The data characterizing Form IV is consistent with a dihydrate.

In some embodiments, Form IV has characteristic XRPD peaks at about 9.2, about 13.4, about 18.5, about 20.0, about 22.6, and about 23.2 degrees 2-theta. In some embodiments, Form IV has at least one characteristic XRPD peak selected from about 9.2, about 13.4, about 18.5, about 20.0, about 22.6, and about 23.2 degrees 2-theta. In some embodiments, Form IV has at least two characteristic XRPD peaks selected from about 9.2, about 13.4, about 18.5, about 20.0, about 22.6, and about 23.2 degrees 2-theta. In some embodiments, Form IV has at least three characteristic XRPD peaks selected from about 9.2, about 13.4, about 18.5, about 20.0, about 22.6, and about 23.2 degrees 2-theta. In some embodiments, Form IV has at least four characteristic XRPD peaks selected from about 9.2, about 13.4, about 18.5, about 20.0, about 22.6, and about 23.2 degrees 2-theta. In some embodiments, Form IV has a characteristic XRPD peak at about 9.2 degrees 2-theta. In some embodiments, Form IV has a characteristic XRPD peak at about 13.4 degrees 2-theta. In some embodiments, Form IV has a characteristic XRPD peak at about 18.5 degrees 2-theta. In some embodiments, Form IV has a characteristic XRPD peak at about 20.0 degrees 2-theta. In some embodiments, Form IV has a characteristic XRPD peak at about 22.6 degrees 2-theta. In some embodiments, Form IV has a characteristic XRPD peak at about 23.2 degrees 2-theta.

In some embodiments, Form IV has at least one characteristic XRPD peak selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta. In some embodiments, Form IV has at least two characteristic XRPD peaks selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta. In some embodiments, Form IV has at least three characteristic XRPD peaks selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta. In some embodiments, Form IV has at least four characteristic XRPD peaks selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta. In some embodiments, Form IV has at least five characteristic XRPD peaks selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta. In some embodiments, Form IV has characteristic XRPD peaks at about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta.

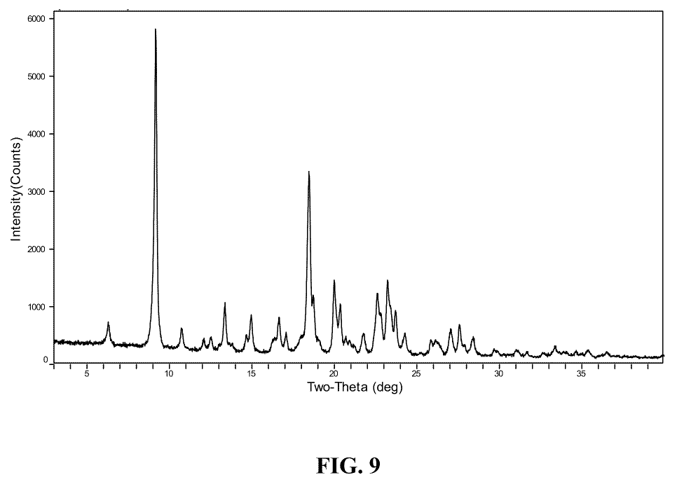

In some embodiments, Form IV has an XRPD pattern with characteristic peaks as substantially shown in FIG. 9.

In some embodiments, Form IV has a DSC thermogram comprising an endotherm onset at a temperature of about 62° C. In some embodiments, Form IV has a DSC thermogram comprising an endotherm peak at a temperature of about 86° C. In some embodiments, Form IV has a DSC thermogram comprising an endotherm onset at a temperature of about 62° C. and an endotherm peak at a temperature of about 86° C. In some embodiments, Form IV has a DSC thermogram comprising an endotherm onset at a temperature of about 189° C. In some embodiments, Form IV has a DSC thermogram comprising an endotherm peak at a temperature of about 192° C. In some embodiments, Form IV has a DSC thermogram comprising an endotherm onset at a temperature of about 189° C. and an endotherm peak at a temperature of about 192° C.

In some embodiments, Form IV shows a weight loss of about 6.8% when heated to about 170° C.

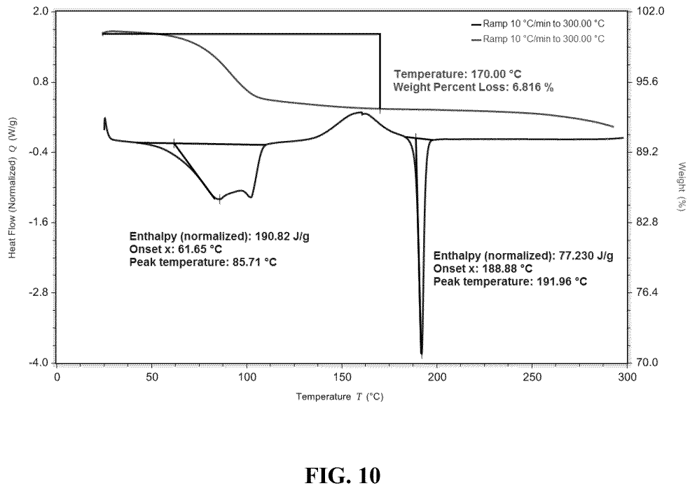

In some embodiments, Form IV has a DSC thermogram substantially as depicted in FIG. 10. In some embodiments, Form IV has a TGA thermogram substantially as depicted in FIG. 10.

In some embodiments, Form IV can be isolated with a crystalline purity of at least about 80%, about 85%, about 90%, about 95%, about 96%, about 97%, about 98%, or about 99%. In some embodiments, Form IV can be isolated with a crystalline purity greater than about 99%. In some embodiments, Form IV can be isolated with a crystalline purity greater than about 99.9%. In some embodiments, Form IV is substantially free of other crystalline form. In some embodiments, Form IV is substantially free of amorphous form.

In some embodiments, Form IV is prepared by precipitating Compound 1 from a solution comprising Compound 1 and S4, wherein S4 is a solvent. In some embodiments, the precipitating is carried out by concentration of the solution, evaporation of S4, reduction of temperature of the solution, addition of anti-solvent, or combination thereof. In some embodiments, S4 is an organic solvent. In some embodiments, S4 is selected from one of the following solvents: water, ethanol, and mixtures thereof. In some embodiments, S4 is a mixture of water and ethanol.

### Compound 1 Form V

In some embodiments, Compound 1 is crystalline and has the characteristics of Form V described below. The data characterizing Form V are consistent with a hydrate.

In some embodiments, Form V has characteristic XRPD peaks at about 6.1, about 9.7, about 12.5, about 18.3, about 21.3, and about 23.3 degrees 2-theta. In some embodiments, Form V has at least one characteristic XRPD peak selected from about 6.1, about 9.7, about 12.5, about 18.3, about 21.3, and about 23.3 degrees 2-theta. In some embodiments, Form V has at least two characteristic XRPD peaks selected from about 6.1, about 9.7, about 12.5, about 18.3, about 21.3, and about 23.3 degrees 2-theta. In some embodiments, Form V has at least three characteristic XRPD peaks selected from about 6.1, about 9.7, about 12.5, about 18.3, about 21.3, and about 23.3 degrees 2-theta. In some embodiments, Form V has at least four characteristic XRPD peaks selected from about 6.1, about 9.7, about 12.5, about 18.3, about 21.3, and about 23.3 degrees 2-theta. In some embodiments, Form V has a characteristic XRPD peak at about 6.1 degrees 2-theta. In some embodiments, Form V has a characteristic XRPD peak at about 9.7 degrees 2-theta. In some embodiments, Form V has a characteristic XRPD peak at about 12.5 degrees 2-theta. In some embodiments, Form V has a characteristic XRPD peak at about 18.3 degrees 2-theta. In some embodiments, Form V has a characteristic XRPD peak at about 21.3 degrees 2-theta. In some embodiments, Form V has a characteristic XRPD peak at about 23.3 degrees 2-theta.

In some embodiments, Form V has at least one characteristic XRPD peak selected from about 6.1, about 9.7, about 12.2, about 12.5, about 15.4, about 18.3, about 18.9, about 19.4, about 21.3, about 22.2, and about 23.3 degrees 2-theta. In some embodiments, Form V has at least two characteristic XRPD peaks selected from about 6.1, about 9.7, about 12.2, about 12.5, about 15.4, about 18.3, about 18.9, about 19.4, about 21.3, about 22.2, and about 23.3 degrees 2-theta. In some embodiments, Form V has at least three characteristic XRPD peaks selected from about 6.1, about 9.7, about 12.2, about 12.5, about 15.4, about 18.3, about 18.9, about 19.4, about 21.3, about 22.2, and about 23.3 degrees 2-theta. In some embodiments, Form V has at least four characteristic XRPD peaks selected from about 6.1, about 9.7, about 12.2, about 12.5, about 15.4, about 18.3, about 18.9, about 19.4, about 21.3, about 22.2, and about 23.3 degrees 2-theta. In some embodiments, Form V has at least five characteristic XRPD peaks selected from about 6.1, about 9.7, about 12.2, about 12.5, about 15.4, about 18.3, about 18.9, about 19.4, about 21.3, about 22.2, and about 23.3 degrees 2-theta. In some embodiments, Form V has characteristic XRPD peaks at about 6.1, about 9.7, about 12.2, about 12.5, about 15.4, about 18.3, about 18.9, about 19.4, about 21.3, about 22.2, and about 23.3 degrees 2-theta.

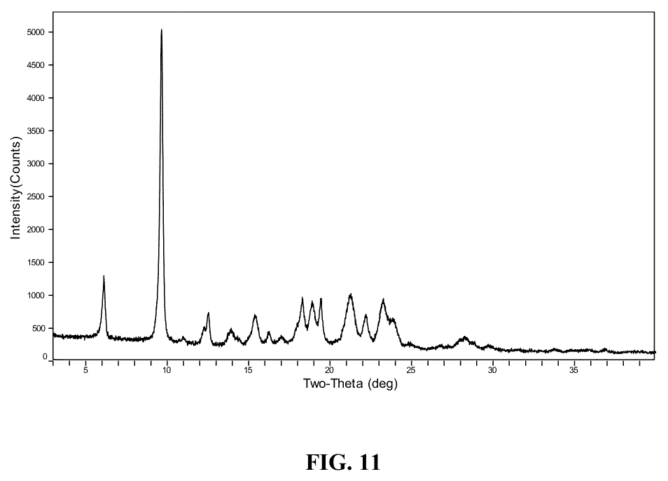

In some embodiments, Form V has an XRPD pattern with characteristic peaks as substantially shown in FIG. 11.

In some embodiments, Form V has a DSC thermogram comprising an endotherm onset at a temperature of about 62° C. In some embodiments, Form V has a DSC thermogram comprising an endotherm peak at a temperature of about 78° C. In some embodiments, Form V has a DSC thermogram comprising an endotherm onset at a temperature of about 62° C. and an endotherm peak at a temperature of about 78° C. In some embodiments, Form V has a DSC thermogram comprising an endotherm onset at a temperature of about 88° C. In some embodiments, Form V has a DSC thermogram comprising an endotherm peak at a temperature of about 96° C. In some embodiments, Form V has a DSC thermogram comprising an endotherm onset at a temperature of about 88° C. and an endotherm peak at a temperature of about 96° C. In some embodiments, Form V has a DSC thermogram comprising an endotherm onset at a temperature of about 189° C. In some embodiments, Form V has a DSC thermogram comprising an endotherm peak at a temperature of about 192° C. In some embodiments, Form V has a DSC thermogram comprising an endotherm onset at a temperature of about 189° C. and an endotherm peak at a temperature of about 192° C.

In some embodiments, Form V shows a weight loss of about 0.4% when heated from about 50° C. to about 110° C.

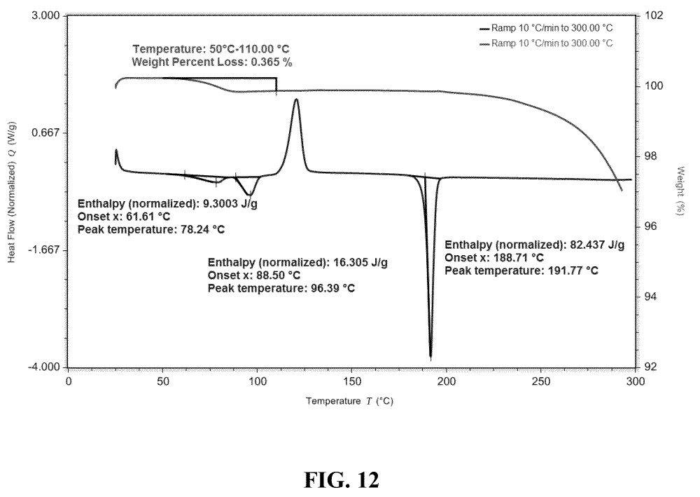

In some embodiments, Form V has a DSC thermogram substantially as depicted in FIG. 12. In some embodiments, Form V has a TGA thermogram substantially as depicted in FIG. 12.

In some embodiments, Form V can be isolated with a crystalline purity of at least about 80%, about 85%, about 90%, about 95%, about 96%, about 97%, about 98%, or about 99%. In some embodiments, Form V can be isolated with a crystalline purity greater than about 99%. In some embodiments, Form V can be isolated with a crystalline purity greater than about 99.9%. In some embodiments, Form V is substantially free of other crystalline form. In some embodiments, Form V is substantially free of amorphous form.

In some embodiments, Form V is prepared by precipitating Compound 1 from a solution comprising Compound 1 and S5, wherein S5 is a solvent. In some embodiments, the precipitating is carried out by concentration of the solution, evaporation of S5, reduction of temperature of the solution, addition of anti-solvent, or combination thereof. In some embodiments, S5 is an organic solvent. In some embodiments, S5 is selected from one of the following solvents: water, 2-Me-THF, and mixtures thereof. In some embodiments, S5 is a mixture of water and 2-Me-THF.

### Compound 1 Form VI

In some embodiments, Compound 1 is crystalline and has the characteristics of Form VI described below. The data characterizing Form VI are consistent with a DMSO solvate.

In some embodiments, Form VI has characteristic XRPD peaks at about 8.9, about 18.0, about 19.0, about 21.1, and about 22.9 degrees 2-theta. In some embodiments, Form VI has at least one characteristic XRPD peak selected from about 8.9, about 18.0, about 19.0, about 21.1, and about 22.9 degrees 2-theta. In some embodiments, Form VI has at least two characteristic XRPD peaks selected from about 8.9, about 18.0, about 19.0, about 21.1, and about 22.9 degrees 2-theta. In some embodiments, Form VI has at least three characteristic XRPD peaks selected from about 8.9, about 18.0, about 19.0, about 21.1, and about 22.9 degrees 2-theta. In some embodiments, Form VI has at least four characteristic XRPD peaks selected from about 8.9, about 18.0, about 19.0, about 21.1, and about 22.9 degrees 2-theta. In some embodiments, Form VI has a characteristic XRPD peak at about 8.9 degrees 2-theta. In some embodiments, Form VI has a characteristic XRPD peak at about 18.0 degrees 2-theta. In some embodiments, Form VI has a characteristic XRPD peak at about 19.0 degrees 2-theta. In some embodiments, Form VI has a characteristic XRPD peak at about 21.1 degrees 2-theta. In some embodiments, Form VI has a characteristic XRPD peak at about 22.9 degrees 2-theta.

In some embodiments, Form VI has at least one characteristic XRPD peak selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta. In some embodiments, Form VI has at least two characteristic XRPD peaks selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta. In some embodiments, Form VI has at least three characteristic XRPD peaks selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta. In some embodiments, Form VI has at least four characteristic XRPD peaks selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta. In some embodiments, Form VI has at least five characteristic XRPD peaks selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta. In some embodiments, Form VI has characteristic XRPD peaks at about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta.

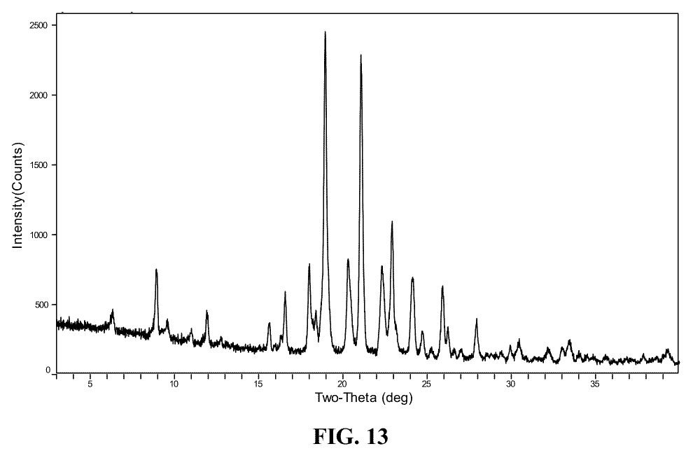

In some embodiments, Form VI has an XRPD pattern with characteristic peaks as substantially shown in FIG. 13.

In some embodiments, Form VI has a DSC thermogram comprising an endotherm onset at a temperature of about 124° C. In some embodiments, Form VI has a DSC thermogram comprising an endotherm peak at a temperature of about 127° C. In some embodiments, Form VI has a DSC thermogram comprising an endotherm onset at a temperature of about 124° C. and an endotherm peak at a temperature of about 127° C. In some embodiments, Form VI has a DSC thermogram comprising an endotherm onset at a temperature of about 186° C.

In some embodiments, Form VI shows a weight loss of about 13.4% when heated to about 190° C.

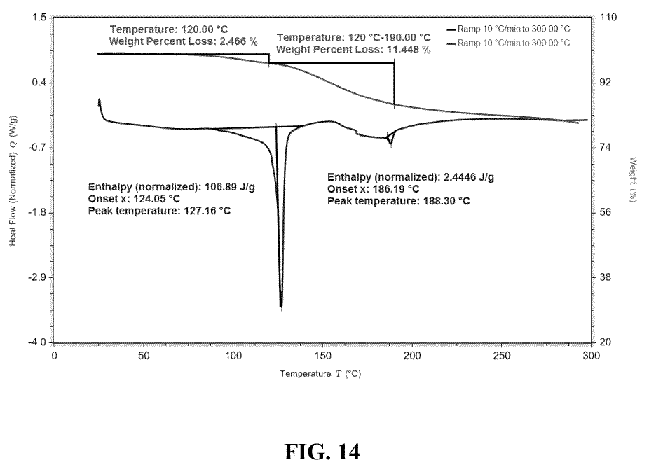

In some embodiments, Form VI has a DSC thermogram substantially as depicted in FIG. 14. In some embodiments, Form VI has a TGA thermogram substantially as depicted in FIG. 14.

In some embodiments, Form VI can be isolated with a crystalline purity of at least about 80%, about 85%, about 90%, about 95%, about 96%, about 97%, about 98%, or about 99%. In some embodiments, Form VI can be isolated with a crystalline purity greater than about 99%. In some embodiments, Form VI can be isolated with a crystalline purity greater than about 99.9%. In some embodiments, Form VI is substantially free of other crystalline form. In some embodiments, Form VI is substantially free of amorphous form.

In some embodiments, Form VI is prepared by precipitating Compound 1 from a solution comprising Compound 1 and S6, wherein S6 is a solvent. In some embodiments, the precipitating is carried out by concentration of the solution, evaporation of S6, reduction of temperature of the solution, addition of anti-solvent, or combination thereof. In some embodiments, S6 is selected from one of the following solvents: water, DMSO, and mixtures thereof. In some embodiments, S5 is a mixture of water and DMSO. In some embodiments, S6 is DMSO.

### Methods of Use

It was found that genetic inactivation of Poly(ADP-Ribose) Polymerase Family Member 14 (PARP14), also referred to as ADP-Ribosyltransferase Diphtheria Toxin-Like 8 (ARTD8) or B Aggressive Lymphoma Protein (BAL2), protected mice against allergen-induced airway disease (Mehrothra et al. 22841009, Cho et al. 23956424), suppressed the infiltration of immune cells such as eosinophils and neutrophils into the lung, and reduced the release of inflammatory Th2 cytokines. In addition, treatment with a PARP14 inhibitor protected mice in a severe asthma model induced by a sensitization and recall challenge with inhaled Alternaria altemata extract. PARP14 inhibitor-treated animals showed a reduced level of airway mucus, blood serum IgE, infiltration of immune cells (eosinophils, neutrophils, and lymphocytes), Th2 cytokines (IL-4, IL-5, and IL13) and alarmins (IL-33 and TSLP) (Ribon internal data).

While not being bound by theory, PARP14 has been shown to affect STAT6 signaling and STAT3 signaling, signaling induced by Th2 cytokines and Th17 cytokines, M1/M2 macrophage polarization, and signaling by lymphocytes. PARP14 has also been shown to be a regulator of Th2/Th17 /THF T cell development, involved in B cell development, and involved in eosinophils/neutrophils recruitment/activation. It is believed that the lymphocytes are likely the ILCs (e.g., ILC2 and ILC3) that get activated by the alarmins (e.g., TSLP and IL-33) and are the main producers of the downstream cytokines (e.g., IL-4, IL-5, and IL-13).

It is suggested that PARP14 inhibition affects the asthma phenotype not only at the level of the second order cytokines (e.g., IL-4, IL-5, and IL-13) and the signaling to the myeloid cells, but that PARP14 inhibition also suppresses the alarmins TSLP and IL-33, which are the key upstream drivers of asthma that get released in response to the allergens.

Compound 1 and solid forms thereof can inhibit the activity of PARP14. For example, Compound 1 and solid forms thereof can be used to inhibit activity of PARP14 in a cell or in an individual or patient in need of inhibition of the enzyme by administering an inhibiting amount of Compound 1 and solid forms thereof to the cell, individual, or patient.

The compounds of the invention can further inhibit the production of IL-10 in a cell. For example, the present invention relates to methods of inhibiting or decreasing the production of IL-10 in a cell by contacting the cell with a PARP14 inhibitor of the invention.

As a PARP14 inhibitor, Compound 1 and solid forms thereof are useful in the treatment of various diseases associated with abnormal expression or activity of PARP14. For example, Compound 1 and solid forms thereof are useful in the treatment of cancer. In some embodiments, the cancers treatable according to the present invention include hematopoietic malignancies such as leukemia and lymphoma. Example lymphomas include Hodgkin’s or non-Hodgkin’s lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett’s lymphoma. Example leukemias include acute lymphocytic leukemia (ALL), acute myelogenous leukemia (AML), chronic lymphocytic leukemia (CLL), and chronic myelogenous leukemia (CML).

Other cancers treatable by the administration of Compound 1 and solid forms thereof include liver cancer (e.g., hepatocellular carcinoma), bladder cancer, bone cancer, glioma, breast cancer, cervical cancer, colon cancer, endometrial cancer, epithelial cancer, esophageal cancer, Ewing’s sarcoma, pancreatic cancer, gallbladder cancer, gastric cancer, gastrointestinal tumors, head and neck cancer, intestinal cancers, Kaposi’s sarcoma, kidney cancer, laryngeal cancer, liver cancer (e.g., hepatocellular carcinoma), lung cancer, prostate cancer, rectal cancer, skin cancer, stomach cancer, testicular cancer, thyroid cancer, and uterine cancer.

In some embodiments, the cancer treatable by administration of Compound 1 and solid forms thereof is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

Compound 1 and solid forms thereof may also have therapeutic utility in PARP14-related disorders in disease areas such as cardiology, virology, neurodegeneration, inflammation, and pain, particularly where the diseases are characterized by overexpression or increased activity of PARP14.

In some embodiments, Compound 1 and solid forms thereof are useful in the treatment of an inflammatory disease. In some embodiments, the inflammatory diseases treatable according to the present invention include inflammatory bowel diseases (e.g., Crohn’s disease or ulcerative colitis), inflammatory arthritis, inflammatory demyelinating disease, psoriasis, allergy and asthma sepsis, allergic airway disease (e.g., asthma), and lupus.

Exemplary inflammatory diseases that are treatable by the disclosed methods include, e.g., asthma, atopic dermatitis, psoriasis, rhinitis, systemic sclerosis, keloids, eosinophilic disorders, pulmonary fibrosis, and other type 2 cytokine pathologies. In some embodiments, the pulmonary fibrosis is idiopathic pulmonary fibrosis.

Eosinophilic disorders that are treatable by the disclosed methods include, e.g., eosinophilic esophagitis (esophagus - EoE), eosinophilic gastritis (stomach - EG), eosinophilic gastroenteritis (stomach and small intestine - EGE), eosinophilic enteritis (small intestine - EE), eosinophilic colitis (large intestine - EC), and eosinophilic chronic rhinosinusitis.

The present invention is further directed, inter alia, to a method of treating or preventing asthma in a patient comprising administering to the patient a therapeutically effective amount of Compound 1 or a solid form thereof.

In some embodiments, the asthma is steroid-insensitive asthma, steroid-refractory asthma, steroid-resistant asthma, atopic asthma, nonatopic asthma, persistent asthma, severe asthma, or steroid-refractory severe asthma. In some embodiments, the severe asthma is T2 high endotype, T2 low endotype, or non-T2 endotype. In some embodiments, the severe asthma is T2 high endotype. In some embodiments, the severe asthma is T2 low endotype or non-T2 endotype. In some embodiments, the severe asthma is T2 low endotype. In some embodiments, the severe asthma is non-T2 endotype.

The present invention further provides a method of:
(a) reducing the level of airway mucus in lung tissue,
(b) reducing blood serum IgE,
(c) reducing immune cell infiltration and activation in bronchoalveolar fluid,
(d) reducing the level of one or more inflammatory cytokines in bronchoalveolar fluid or in lung tissue, or
(e) reducing the level of one or more alarmins in bronchoalveolar fluid or lung tissue,
   in a patient, where the method comprises administering to the patient Compound 1 or a solid form thereof.

In some embodiments, the present invention provides a method of reducing the level of airway mucus in lung tissue in a patient.

In some embodiments, the present invention provides a method of reducing immune cell infiltration and activation in bronchoalveolar fluid in a patient. In some embodiments, the immune cells are eosinophils, neutrophils, or lymphocytes.

In some embodiments, the present invention provides a method of reducing one or more inflammatory cytokines in bronchoalveolar fluid or in lung tissue in a patient. In some embodiments, the inflammatory cytokine is a Th2 cytokine or Th17 cytokine. In some embodiments, the inflammatory cytokine is a Th2 cytokine. In some embodiments, the inflammatory cytokine is IL-4, IL-5, IL13, or IL-17A. In some embodiments, the inflammatory cytokine is IL-4, IL-5, or IL 13.

In some embodiments, the present invention provides a method of reducing an alarmin in bronchoalveolar fluid or in lung tissue in a patient. In some embodiments, the alarmin is IL-25, IL-33 or TSLP.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PARP14 or “contacting” a cell with Compound 1 includes the administration of Compound 1 or a solid form thereof to an individual or patient, such as a human, having PARP14, as well as, for example, introducing a compound of the invention into a sample containing a cellular or purified preparation containing PARP14.

As used herein, the term “individual” or “patient,” used interchangeably, refers to mammals, and particularly humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease.

As used herein, the term “reducing” is with respect to the level in the patient prior to administration. More specifically, when a biomarker or symptom is reduced in a patient, the reduction is with respect to the level of or severity of the biomarker or symptom in the patient prior to administration of Compound 1 or a solid form thereof.

### Combination Therapies

One or more additional pharmaceutical agents or treatment methods such as, for example, chemotherapeutics or other anti-cancer agents, immune enhancers, immunosuppressants, immunotherapies, radiation, anti-tumor and anti-viral vaccines, cytokine therapy (e.g., IL2, GM-CSF, etc.), and/or kinase (tyrosine or serine/threonine), epigenetic or signal transduction inhibitors can be used in combination with Compound 1 and solid forms thereof. The agents can be combined with Compound 1 and solid forms thereof in a single dosage form, or the agents can be administered simultaneously or sequentially as separate dosage forms.

Suitable agents for use in combination with Compound 1 and solid forms thereof for the treatment of cancer include chemotherapeutic agents, targeted cancer therapies, immunotherapies or radiation therapy. Compound 1 and solid forms thereof may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with Compound 1 and solid forms thereof. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with Compound 1 and solid forms thereof. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other anti-cancer agent(s) include antibody therapeutics to checkpoint or costimulatory molecules such as CTLA-4, PD-1, PD-L1 or 4-1BB, respectively, or antibodies to cytokines (IL-10, TGF-β, etc.). Exemplary cancer immunotherapy antibodies include pembrolizumab, ipilimumab, nivolumab, atezolizumab and durvalumab. Additional anti-cancer agent(s) include antibody therapeutics directed to surface molecules of hematological cancers such as ofatumumab, rituximab and alemtuzumab.

Methods for the safe and effective administration of most of these chemotherapeutic agents are known to those skilled in the art. In addition, their administration is described in the standard literature. For example, the administration of many of the chemotherapeutic agents is described in the “Physicians’ Desk Reference” (PDR, e.g., 1996 edition, Medical Economics Company, Montvale, NJ), the disclosure of which is incorporated herein by reference as if set forth in its entirety.

### Formulation, Dosage Forms and Administration

When employed as pharmaceuticals, Compound 1 and solid forms thereof can be administered in the form of pharmaceutical compositions. A pharmaceutical composition refers to a combination of Compound 1 and solid forms thereof, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of Compound 1 and solid forms thereof in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient’s symptoms, and the like.

For preparing solid compositions such as tablets, the principal active ingredient is mixed with a pharmaceutical excipient to form a solid pre-formulation composition containing a homogeneous mixture of Compound 1 and solid forms thereof. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which Compound 1 and solid forms thereof and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of Compound 1 and solid forms thereof can vary according to, for example, the particular use for which the treatment is made, the manner of administration of Compound 1 and solid forms thereof, the health and condition of the patient, and the judgment of the prescribing physician. The proportion or concentration of a Compound 1 in a pharmaceutical composition can vary depending upon a number of factors including dosage, chemical characteristics (e.g., hydrophobicity), and the route of administration. For example, Compound 1 and solid forms thereof can be provided in an aqueous physiological buffer solution containing about 0.1 to about 10% w/v of Compound 1 and solid forms thereof for parenteral administration. Some typical dose ranges are from about 1 µg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, formulation of the excipient, and its route of administration. Effective doses can be extrapolated from dose-response curves derived from in vitro or animal model test systems.

Compound 1 and solid forms thereof can also be formulated in combination with one or more additional active ingredients which can include any pharmaceutical agent such as anti-viral agents, anti-cancer agents, vaccines, antibodies, immune enhancers, immune suppressants, anti-inflammatory agents and the like.

### EXAMPLES

### General Experimental

### X-Ray Powder Diffraction (XRPD)

XRPD patterns were identified with an X-ray diffractometer (PANalytical Empyrean). The system was equipped with PIXcel^{1D} detector. Samples were scanned from 3 to 40° 2θ, at a step size of 0.013° 2θ. The tube voltage and current were 45 KV and 40 mA, respectively.

### Differential Scanning Calorimeter (DSC)

DSC was performed using a Discovery DSC 250 (TA Instruments, US). The sample was placed into an aluminum pin-hole hermetic pan and the weight was accurately recorded. Then the sample was heated at a rate of 10° C./min from 25° C. to the final temperature.

### Thermogravimetric Analysis (TGA)

TGA was carried out on a Discovery TGA 55 (TA Instruments, US). The sample was placed into an open tared aluminum pan, automatically weighed, and inserted into the TGA furnace. The sample was heated at a rate of 10° C./min from room temperature (RT, about 24° C.) to the final temperature.

### Polarized Light Microscopy (PLM)

Light microscopy was performed using a Polarizing Microscope ECLIPSE LV100POL (Nikon, JPN).

### High Performance Liquid Chromatography (HPLC)

HPLC analysis was performed with an Agilent HPLC 1260 series instrument. HPLC method for solubility and stability testing is listed in the table below:

| | |
|---|---|
| Column | ACE Excel 3 C18 4.6^{∗}150 mm 3.0 µm |
| Mobile Phase | A: 5 mM NH₄COOH in water |
| | B: Acetonitrile (CAN) |
| Gradient (T/B%) | 0/10, 13/90, 16/90, 16.5/10, 20/10 |
| Column Temperature | 40° C. |
| Detector | DAD; 243 nm |
| Flow Rate | 1.0 mL/min |
| Injection Volume | 4 µL |
| Run Time | 20 mins |
| Post Time | 0 min |
| Diluent | ACN: Water=1: 1 (v: v) |

### Liquid Chromatography Mass Spectrometry (LCMS)

LCMS was performed using an Agilent HPLC 1260 series instrument equipped with mass spectrometry detector. LCMS method for testing is listed in the following table:

| | |
|---|---|
| Column | SB-C18 4.6^{∗}50 mm^{∗}1.8 µm |
| Mobile Phase | A: 0.1% Formic Acid (FA) in water |
| | B: 0.1% FA in ACN |
| Gradient (T/B%) | 0/10, 4/100, 6/100, 6.1/10 |
| Column Temperature | 40° C. |
| Detector | DAD; 254 /210/192/220 nm |
| Flow Rate | 1.0 mL/min |
| Injection Volume | 5 µL |
| Run Time | 8.1 min |
| Post Time | 2.0 min |
| Diluent | ACN: Water=1: 1 (v: v) |

### Particle Size Distribution (PSD)

PSD analysis was carried out on Malvern Mastersizer 3000 Particle Size Analyzer (NBQC-MAS-1). Appropriate amount of sample was pre-dispersed by Water. The mixture was shaken and mixed completely by an IKA mixer. After the measurement of background, appropriate volume of suspension was added to 450~500 mL of water until the obscuration reached appropriate range. Then the PSD were measured three times and the average value was adopted. The detailed parameters are listed as below.

Sample disperse unit: Hydro EV used for wet method.
Stirrer Speed: 25000 rpm
Models: General purpose
Lower/Upper Limit of Obscuration: 5%-20%
Ultrasound percentage: 60%
Background Measurement Time: 12 s
Sample Measurement Time: 12 s
Measurement Cycles: 3
Delay between measurement duration: 0 s

### Example 1. Synthesis of Compound 1

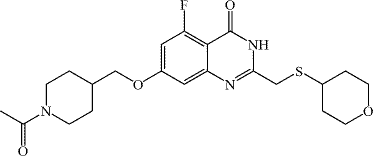 [SMILES: CC(=O)N1CCC(COc2cc(F)c3c(=O)[nH]c(CSC4CCOCC4)nc3c2)CC1]

### Step 1: 1-(4-(Hydroxymethyl)Piperidin-1-yl)Ethan-1-One

Acetic anhydride (558.42 g, 5469.89 mmol, 1.05 equiv) was added dropwise to the solution of piperidin-4-ylmethanol (600.00 g, 5209.42 mmol, 1.00 equiv) in ethanol (2.60 L) slowly at room temperature. The resulting solution was stirred overnight at room temperature, and then the resulting mixture was concentrated under vacuum. The pH value of the solution was adjusted to 8 with saturated Na₂CO₃ aqueous solution. The resulting solution was extracted with 15×2.5 L of dichloromethane. The organic layers were combined and concentrated to afford 740 g (90.36%) of title compound as light brown oil. LCMS: [M+H]⁺ 158.10.

### Step 2: (1-Acetylpiperidin-4-yl)Methyl 4-Methylbenzenesulfonate

To a solution of 1-(4-(hydroxymethyl)piperidin-1-yl)ethan-1-one (740.00 g, 4706.99 mmol, 1.00 equiv), Triethylamine (TEA) (952.60 g, 9413.94 mmol, 2.0 equiv) and 4-dimethylaminopyridine (DMA)P (57.50 g, 470.70 mmol, 0.10 equiv) in toluene (9.00 L) was slowly added p-toluenesulfonyl chloride (TsCl) (987.07 g, 5177.69 mmol, 1.10 equiv) at 10° C. in a water/ice bath, and then the resulting solution was stirred at room temperature for 8 h. Thin layer chromatography (TLC) indicated some 1-(4-(hydroxymethyl)piperidin-1-yl)ethan-1-one remained. The solids were filtered out and another batch of DMAP (28.8 g, 236 mmol, 0.05 equiv), TEA (190.8 g, 1889 mmol, 0.4 equiv) and TsCl (125.6 g, 661 mmol, 0.14 equiv) was added to the resulting solution. The resulting solution was stirred for another 15 hours at room temperature. TLC indicated some starting materials still remained. The solids were filtered out and a third batch of DMAP (23 g, 188.5 mmol, 0.04 equiv), TEA (95.4 g, 944.5 mmol, 0.2 equiv) and TsCl (89.8 g, 472.6 mmol, 0.1 equiv) was added to the resulting solution. The resulting solution was stirred for another 22 hours at room temperature. TLC showed the reaction completed. The solids were filtered out, DMAP (172.5 g, 1413.9 mmol, 0.3 equiv) was added into the solution to react with the excess TsCl for 1 hour at room temperature, providing insoluble white solids which were removed by filtration. The filtrate was concentrated under vacuum and dissolved in 4 L of ethyl acetate, which was washed with 1×2 L of HCl (0.5 M, 0.2 eq), 2×1 L saturated NaHCO₃ aqueous solution and 2×1 L brine. The organic phase was dried over anhydrous sodium sulfate and concentrated to afford 1.25 kg (85.28%) of the title compound as a brown oil. LCMS: [M+H]⁺ 312.15.

### Step 3: S-(Tetrahydro-2H-Pyran-4-yl) Ethanethioate

A solution of 1-(potassiosulfanyl)ethanone (499.34 g, 4372.54 mmol, 1.10 equiv) was added to a solution of 4-bromooxane (656.00 g, 3975.04 mmol, 1.00 equiv) in dimethylformamide (DMF) (1.40 L). The mixture was stirred overnight at 45° C. The solids were filtered out. The filtrate was concentrated under vacuum to remove ~1 L DMF. The crude product was dissolved in 3 L of brine. The resulting solution was extracted with 3×3 L of ethyl acetate. The organic layers were combined, dried over anhydrous sodium sulfate and concentrated under vacuum to afford 472 g (74.11%) of the title compound as a brown oil.

### Step 4: 2,6-Difluoro-4-Hydroxybenzoic Acid

A solution of 2,6-difluoro-4-hydroxybenzonitrile (2.00 kg, 12894.57 mmol, 1.00 equiv), potassium hydroxide (2.39 kg, 42552.09 mmol, 3.30 equiv) in water (8.40 L) was stirred for overnight at 100° C. After completion, the resulting solution was cooled to room temperature and the pH value was adjusted to 3 with HCl (12 M). The precipitated solids were collected by filtration and dried under oven to afford 2.09 kg (93.10%) of the title compound as a white solid. LCMS: [M-H]⁺ 173.00

### Step 5: Methyl 2,6-Difluoro-4-hydroxybenzoate

A solution of 2,6-difluoro-4-hydroxybenzoic acid (2.09 kg, 12004.39 mmol, 1.00 equiv) and sulfuric acid (1.63 L) in methanol (10 L) was stirred for 2 days at 70° C. After completion, the resulting solution was concentrated to remove most of the methanol. The precipitated solids were collected by filtration, which was slurried in water for 30 minutes. The solids were collected by filtration and oven-dried to afford 1.8 kg (79.70%) of the title compound as a white solid. LCMS: [M+H]⁺ 189.00.

### Step 6: Methyl 4-((1-Acetylpiperidin-4-yl)Methoxy)-2,6-Difluorobenzoate

A solution of methyl 2,6-difluoro-4-hydroxybenzoate (628.00 g, 3338.12 mmol, 1.00 equiv), (1-acetylpiperidin-4-yl)methyl 4-methylbenzenesulfonate (1247.39 g, 4005.74 mmol, 1.20 equiv), and K₂CO₃ (922.69 g, 6676.23 mmol, 2.00 equiv) in 2-butanone (6.0 L) was stirred overnight at 80° C. After completion, K₂CO₃ was removed by filtration. The filtrate was concentrated to remove most of the2-butanone and the product was precipitated. After addition of a mixed solvent (a 2:3 mixture of ethyl acetate: petroleum ether) and stirring for 30 minutes, the solids were collected by filtration and oven-dried to afford 980 g (89.69%) of the title compound as a white solid. LCMS: [M+H]⁺328.05

### Step 7: Methyl 4-((1-Acetylpiperidin-4-yl)Methoxy)-2-((2,4-Dimethoxybenzyl)Amino)-6-Fluorobenzoate

A solution of methyl 4-[(1-acetylpiperidin-4-yl)methoxy]-2,6-difluorobenzoate (750.00 g, 2291.28 mmol, 1.00 equiv), 1-(2,4-dimethoxyphenyl)methanamine (459.74 g, 2749.54 mmol, 1.20 equiv), and K₂CO₃ (633.33 g, 4582.56 mmol, 2.00 equiv) in N-methyl pyrrolidone (3.90 L) was stirred overnight at 80° C. The resulting solution was diluted with 5 L of water. The resulting solution was extracted with 3×8 L of ethyl acetate. The organic phase was washed with 3 ×3 L of brine, which was dried over anhydrous sodium sulfate and concentrated under vacuum. The crude product was stirred in mixed solvent (a 1:1 mixture of ethyl acetate:petroleum ether) for 1 hour. The solids were collected by filtration and dried under oven to afford 880 g (80.94%) of the title compound as a white solid.

### Step 8: 7-((1-Acetylpiperidin-4-yl)Methoxy)-2-(Chloromethyl)-5-Fluoroquinazolin-4(3H)-One

A solution of methyl 4-[(1-acetylpiperidin-4-yl)methoxy]-2-[[(2,4-dimethoxyphenyl)methyl]amino]-6-fluorobenzoate (800.00 g, 1685.88 mmol, 1.00 equiv), chloroacetonitrile (381.85 g, 5057.65 mmol, 3.00 equiv) and HCl(gas)/1,4-dioxane (4 M, 2.60 L) was stirredovernight at 40° C. After completion, the resulting solution was cooled to room temperature and the precipitated solids were collected by filtration. Then the solids were slurried in 1.5 L of acetonitrile. The solids were collected by filtration and slurried in 1.5 L of ethyl acetate. The solids were collected by filtration and slurried in 1.5 L acetonitrile at 50° C. The solids were collected by filtration and oven-dried. This resulted in 650 g (104.82%) of the title compound as an off-white solid. LCMS: [M+H]⁺ 368.05.

### Step 9: 7-((1-Acetylpiperidin-4-yl)Methoxy)-5-Fluoro-2-(((Tetrahydro-2H-Pyran-4-yl)Thio)Methyl)Quinazolin-4(3H)-One

A solution of S-(tetrahydro-2H-pyran-4-yl) ethanethioate (0.61 kg, 3806.31 mmol, 1.00 equiv) and NaOH (305.0 g, 7629.4 mmol, 2.00 equiv) in 3.0 L water was added to a solution of 7-[(1-acetylpiperidin-4-yl)methoxy]-2-(chloromethyl)-5-fluoro-3H-quinazolin-4-one (1.40 kg, 3806.31 mmol, 1.00 equiv) and NaOH (305.0 g, 7629.4 mmol, 2.00 equiv) in 4.6 L H₂O at 0° C. The resulting solution was stirred for 3 hours at room temperature. After completion, the resulting solution was diluted with 22 L of water, and the solids were filtered out. The pH value of the filtrate was adjusted to 5 with concentrated HCl (12 mol/L). The precipitated solids were collected by filtration. The solids were slurried in 2.5 L of acetonitrile. The solids were collected by filtration. The crude product was purified by recrystallization from methanol (7 L, reflux at 80° C.). The solids were collected by filtration and oven-dried to afford 750 g (43.83%) of title compound as an off-white solid. All the mother liquors were combined and concentrated, and the crude product was purified by recrystallization from methanol (2 L, reflux at 80° C.) two times. The solids were collected by filtration and oven-dried to afford another 150 g of title compound as an off-white solid, which was identified as Form I. LCMS: [M+H]⁺ 450.10. ¹H NMR (400 MHz, DMSO-d₆) δ 12.17 (s, 1H), 6.90 (d, J =1.2 Hz, 1H, 6.87 (d, J =1.5 Hz, 1H), 4.40 (d, J = 12.8 Hz, 1H), 4.10-3.90 (m, 2H), 3.89 – 3.77 (m, 3H), 3.59 (s, 2H), 3.38-3.28 (m, 2H), 3.10 - 3.00 (m, 2H), 2.60 - 2.57 (m, 1H), 2.10 - 2.00 (m, 4H), 1.98 - 1.89 (m, 2H), 1.84 – 1.72 (m, 2H), 1.50-1.40 (m, 2H), 1.42-1.05 (m, 2H). Additional characterization of Form I is provided in Example 7.

### Example 2. Preliminary Solubility Testing

The preliminary solubility of Compound 1 Form I was estimated via visual assessment. Results are shown in the table below:

TABLE 1

Estimated Solubility of Compound 1 Form I

| No. | Solvent | Solubility (mg/mL) | No. | Solvent | Solubility (mg/mL) |
|---|---|---|---|---|---|
| 1 | Methanol (MeOH) | 10.0 | 9 | Heptane | <0.6 |
| 2 | Ethanol (EtOH) | 3.3 | 10 | DCM | 6.3 |
| 3 | Isopropyl alcohol (IPA) | <0.6 | 11 | 2-Me-THF | 10.0 |
| 4 | Methyl ethyl ketone (MEK) | 10.0 | 12 | THF | 4.2 |
| 5 | Acetone | 1.0 | 13 | ACN | <0.6 |
| 6 | Methyl tert-butyl ether (MTBE) | <0.6 | 14 | DMSO | 16.7 |
| 7 | Ethyl acetate (EA) | 2.5 | 15 | Toluene | <0.6 |
| 8 | Isopropyl acetate (IPAC) | 1.3 | 16 | Water | <0.6 |

Values are rounded to nearest whole number and reported as “<” if dissolution was not observed, and as “>” if dissolution occurred after addition of first aliquot.

### Example 3. Evaporative Crystallization in Single and Mixed Solvents Screen

About 10 mg of Compound 1 (as prepared in Example 1) was dissolved in single or mixed solvents. The drug solutions were filtered. Filtrates were then used for slow evaporation at room temperature. Solid samples were collected and analyzed by XRPD after air drying. If new XRPD patterns were identified, the sample was further analyzed by DSC and TGA.

TABLE 2

Results of Evaporative Crystallization in Single/Mixed Solvents

| No. | Solvent | V₁/ V₂ (mL) | Results |
|---|---|---|---|
| 1 | MeOH | 1.0 | Form I |
| 2 | MEK | 1.0 | Form II |
| 3 | Dichloromethane (DCM) | 1.5 | Form I |
| 4 | Tetrahydrofuran (THF) | 1.2 | Pattern A |
| 5 | 2-methyltetrahydrofuran (2-Me-THF) | 2.5 | Pattern B |
| 6 | MeOH/Water | 0.5/1.0 | Amorphous |
| 7 | MeOH/Acetone | 0.4/0.4 | Amorphous |
| 8 | MeOH/MTBE | 2.0/0.1 | Form I |
| 9 | MeOH/EA | 0.4/0.4 | Amorphous |
| 10 | MeOH/ACN | 0.8/0.4 | Amorphous |
| 11 | THF/Water | 0.4/0.1 | Sticky |
| 12 | THF/MeOH | 0.2/0.2 | Pattern C |
| 13 | THF/Acetone | 1.0/0.4 | Pattern A |
| 14 | THF/IPAc | 1.2/0.2 | Pattern A |
| 15 | THF/DCM | 1.5/1.0 | Pattern A+ Form I |

Samples of Patterns A, B, and C were not further isolated or characterized.

### Example 4. Slurry Study

About 30 mg of Compound 1 (as prepared in Example 1) was added into a single solvent to provide a suspension. The suspensions were kept stirring at RT for 3 days. Solid samples were collected by filtration and analyzed by XRPD after stirring. If new XRPD patterns were identified, the sample was further analyzed by DSC and TGA.

TABLE 3

Results of Slurry in Single Solvent

| No. | Solvent | Loading Conc. (mg/mL) | Result | |
|---|---|---|---|---|
| | | | Wet cake | After drying |
| 1 | EtOH | 30 | Form I | Form I |
| 2 | Acetone | 30 | Form I | Form I |
| 3 | MTBE | 30 | Form II^{∗} | Form II^{∗} |
| 4 | IPAc | 30 | Form II | Form II |
| 5 | ACN | 30 | Form III | Form I |
| 6 | DCM | 75 | Similar with Form III | Form I |
| 7 | 2-Me-THF | 75 | Form II | Form II |

^{∗}Represents that an extra diffraction peak at 18° (2 θ was observed.

Alternatively, about 30 mg of Compound 1 (as prepared in Example 1) was added into single solvent to provide a suspension. The suspensions were kept stirring at 50° C. for 3 days. Solid samples were collected by filtration and analyzed by XRPD after stirring. If new XRPD patterns were identified, the sample was further analyzed by DSC and TGA.

TABLE 4

Results of Slurry in Single Solvent

| No. | Solvent | Loading Conc. (mg/mL) | Result | |
|---|---|---|---|---|
| | | | Wet cake | After drying |
| 1 | IPA | 75 | Form I + Form II | Form I + Form |
| | | | | II |
| 2 | Toluene | 60 | Form II | Form II |
| 3 | Water | 60 | Form IV | Form V |

### Example 5. Cooling Crystallization Screen

About 20 mg of Compound 1 (as prepared in Example 1) was weighed into vials and the selected solvents were added to provide a nearly clear solution with heating. The suspensions were filtered to obtain saturated drug solutions. The saturated drug solutions were cooled to the indicated final temperature directly. Any solids obtained were characterized accordingly. Results are shown below in Table 5.

TABLE 5

Results of Cooling Crystallization in Single Solvent

| No. | Solvent | Initial temp | Final temp | Volume (mL) | Result | |
|---|---|---|---|---|---|---|
| | | | | | Wet cake | After drying |
| 1 | EtOH | 50° C. | 26° C. | 1.0 | Form II | / |
| 2 | Acetone | | | 3.0 | Form I | Form I |
| 3 | EA | | | 1.2 | Form II | / |
| 4 | IPAc | | 6° C. | 3.0 | Form II | / |
| 5 | ACN | | 26° C. | 2.8 | Form III | Form I |

About 20 mg of Compound 1 (as prepared in Example 1) was weighed into vials and the selected mixed solvents were added to make a nearly clear solution with heating. The suspensions were filtered to obtain a saturated drug solution. The saturated drug solutions were cooled to the indicated final temperature directly. Any solids obtained were characterized accordingly.

TABLE 6

Results of Cooling Crystallization in Mixed Solvents

| No. | Solvent | Initial temp | Final temp | Volume (mL) | Result | |
|---|---|---|---|---|---|---|
| | | | | | Wet cake | After drying |
| 1 | EtOH/Water | 50° C. | -20° C. | 0.5/0.2 | Form IV | / |
| 2 | EtOH/Acetone | | 26° C. | 0.5/0.2 | Form I | Form I |
| 3 | MeOH/ACN | | | 0.2/0.2 | Form III | Form I |
| 4 | EtOH/EA | | 6° C. | 0.4/0.4 | Form II | / |
| 5 | 2-Me-THF/Acetone | | 26° C. | 0.4/0.4 | Form I | Form I |
| 6 | 2-Me-THF/ACN | | | 0.5/0.6 | Form I | Form I |
| 7 | 2-Me-THF/EtOH | | 6° C. | 0.4/0.4 | Form II | / |
| 8 | 2-Me-THF/Water | | | 0.4/0.2 | Form V | Form V |
| 9 | Acetone/EA | | 26° C. | 0.4/0.6 | Form I | Form I |
| 10 | Acetone/Water | | | 0.4/0.1 | Form I | Form I |
| 11 | ACN/Water | | | 0.4/0.1 | Form III | Form I |

### Example 6. Anti-Solvent Precipitation Screen

About 20 mg of Compound 1 (as prepared in Example 1) was weighed into vials and the selected solvents were added to make saturated solution. After filtration, anti-solvents were added into the filtrates gradually at RT. If precipitation occurred, products were characterized accordingly.

TABLE 7

Results of Anti-solvent Precipitation

| No. | Solvent | Anti-solvent | V₁/ V₂(mL) | Result |
|---|---|---|---|---|
| 1 | MeOH | MTBE | 2.0/6.0 | Solution |
| 2 | MEK | ACN | 1.5/2.0 | Form III |
| 3 | Acetone | MTBE | 2.0/2.0 | Form I |
| 4 | EA | MTBE | 1.0/3.0 | Form II |
| 5 | DCM | Heptane | 1.0/2.0 | Form III |
| 6 | THF | Heptane | 0.5/2.0 | Form II |
| 7 | 2-Me-THF | Toluene | 1.0/3.0 | Solution |
| 8 | Dimethyl solfoxide (DMSO) | Water | 0.2/1.0 | Form VI |
| 9 | EtOH | MTBE | 1.0/3.0 | Solution |
| 10 | 2-Me-THF | Isopropyl ether (IPE) | 1.0/2.0 | Form II |

### Example 7. Characterization of Form I

The XRPD pattern of Form I is shown in FIG. 1 and the peak data is given below in Table 8.

TABLE 8

XRPD Peak Data for Form I

| 2-Theta | Height |
|---|---|
| 6.3 | 3338 |
| 8.9 | 13400 |
| 9.4 | 583 |
| 10.3 | 101 |
| 10.8 | 399 |
| 11.2 | 415 |
| 12.5 | 1199 |
| 12.9 | 292 |
| 13.6 | 94 |
| 14.3 | 1147 |
| 14.7 | 317 |
| 15.3 | 1089 |
| 16.2 | 226 |
| 16.9 | 2546 |
| 17.7 | 893 |
| 18.0 | 447 |
| 18.6 | 154 |
| 19.0 | 1144 |
| 19.6 | 57 |
| 20.1 | 728 |
| 20.5 | 660 |
| 21.3 | 364 |
| 21.7 | 191 |
| 21.9 | 235 |
| 22.4 | 3777 |
| 22.8 | 271 |
| 23.1 | 121 |
| 23.5 | 894 |
| 23.8 | 1096 |
| 24.1 | 668 |
| 24.6 | 68 |
| 25.1 | 786 |
| 25.7 | 810 |
| 26.2 | 230 |
| 26.8 | 372 |
| 27.8 | 197 |
| 28.7 | 296 |
| 29.3 | 92 |
| 30.1 | 186 |
| 30.6 | 257 |
| 31.3 | 115 |
| 32.7 | 204 |
| 33.5 | 160 |
| 34.1 | 129 |
| 37.2 | 131 |
| 38.2 | 186 |
| 38.6 | 134 |
| 39.4 | 134 |

Form I exhibits a DSC thermogram having an endotherm with an onset temperature at about 82° C. and a peak at a temperature of about 92° C.; and an endotherm with an onset temperature at about 189° C. and a peak at a temperature of about 192° C. Form I shows a weight loss of about 1.1% when heated to 110° C. FIG. 2 shows a DSC thermogram and a TGA thermogram of Compound 1 Form I. The data suggest that Form I may be a hydrate. FIG. 3 shows the PLM micrograph of Form I.

### Example 8. Characterization of Form II

The XRPD pattern of Form II is shown in FIG. 4 and the peak data is given below in Table 9.

TABLE 9

XRPD Peak Data for Form II

| 2-Theta | Height |
|---|---|
| 8.6 | 1253 |
| 9.4 | 1882 |
| 12.2 | 501 |
| 14.2 | 61 |
| 14.8 | 712 |
| 15.9 | 778 |
| 16.1 | 857 |
| 17.4 | 390 |
| 17.7 | 626 |
| 18.1 | 569 |
| 19.5 | 1456 |
| 20.0 | 947 |
| 20.7 | 392 |
| 20.9 | 490 |
| 22.7 | 2399 |
| 23.6 | 83 |
| 24.2 | 135 |
| 24.6 | 853 |
| 25.2 | 270 |
| 25.4 | 217 |
| 25.8 | 42 |
| 26.2 | 93 |
| 27.1 | 395 |
| 27.8 | 59 |
| 28.3 | 414 |
| 29.8 | 70 |
| 30.9 | 78 |
| 31.5 | 86 |
| 32.0 | 101 |
| 32.5 | 63 |
| 33.6 | 102 |
| 34.1 | 71 |
| 34.8 | 67 |
| 35.5 | 42 |
| 36.7 | 38 |
| 37.0 | 33 |
| 37.6 | 50 |
| 39.4 | 39 |

Form II exhibits a DSC thermogram having an endotherm with an onset temperature at about 189° C. and a peak at a temperature of about 190° C. Form II shows no weight loss when heated to 150° C. FIG. 5 shows a DSC thermogram and a TGA thermogram of Compound 1 Form II. The data suggest that Form II may be an anhydrous crystalline form. FIG. 6 shows the PLM micrograph of Form II.

### Example 9. Characterization of Form III

The XRPD pattern of Form III is shown in FIG. 8 and the peak data is given below in Table 10.

TABLE 10

XRPD Peak Data for Form III

| 2-Theta | Height |
|---|---|
| 6.2 | 1257 |
| 8.9 | 7102 |
| 9.4 | 68 |
| 10.0 | 137 |
| 11.0 | 195 |
| 12.3 | 737 |
| 12.5 | 135 |
| 13.0 | 78 |
| 14.2 | 676 |
| 14.6 | 199 |
| 15.5 | 495 |
| 16.2 | 251 |
| 16.9 | 1848 |
| 18.1 | 870 |
| 18.5 | 108 |
| 19.0 | 1151 |
| 20.2 | 265 |
| 20.4 | 357 |
| 20.7 | 277 |
| 21.5 | 754 |
| 21.9 | 1893 |
| 22.6 | 300 |
| 23.0 | 595 |
| 23.6 | 311 |
| 23.8 | 613 |
| 24.1 | 344 |
| 24.8 | 456 |
| 25.3 | 192 |
| 26.0 | 263 |
| 26.7 | 237 |
| 27.5 | 66 |
| 28.1 | 49 |
| 28.5 | 190 |
| 30.5 | 218 |
| 32.6 | 117 |
| 33.3 | 81 |
| 33.6 | 84 |
| 34.2 | 63 |
| 36.1 | 70 |
| 37.7 | 71 |
| 38.9 | 89 |
| 39.4 | 79 |

### Example 10. Characterization of Form IV

The XRPD pattern of Form IV is shown in FIG. 9 and the peak data is given below in Table 11.

TABLE 11

XRPD Peak Data for Form IV

| 2-Theta | Height |
|---|---|
| 6.3 | 394 |
| 9.2 | 5553 |
| 10.7 | 359 |
| 12.1 | 197 |
| 12.5 | 220 |
| 13.4 | 826 |
| 13.8 | 130 |
| 14.6 | 293 |
| 15.0 | 644 |
| 16.3 | 216 |
| 16.7 | 586 |
| 17.1 | 316 |
| 18.0 | 181 |
| 18.5 | 3132 |
| 18.7 | 952 |
| 19.1 | 212 |
| 20.0 | 1177 |
| 20.4 | 836 |
| 20.7 | 265 |
| 20.9 | 107 |
| 21.8 | 322 |
| 22.6 | 1019 |
| 22.8 | 676 |
| 23.2 | 1199 |
| 23.7 | 766 |
| 24.3 | 290 |
| 25.8 | 254 |
| 26.1 | 251 |
| 27.0 | 407 |
| 27.6 | 487 |
| 27.9 | 133 |
| 28.4 | 287 |
| 29.7 | 120 |
| 31.0 | 111 |
| 31.7 | 83 |
| 32.6 | 61 |
| 33.4 | 176 |
| 34.7 | 92 |
| 35.4 | 103 |
| 36.5 | 104 |

Form IV exhibits a DSC thermogram having an endotherm with an onset temperature of about 62° C. and a peak at a temperature of about 86° C.; and an endotherm with an onset temperature at about 189° C. and a peak at a temperature of about 192° C. Form IV shows a weight loss of about 6.8% when heated to 170° C. FIG. 10 shows a DSC thermogram and a TGA thermogram of Compound 1 Form IV. The data suggest that Form IV may be a dihydrate.

### Example 11. Characterization of Form V

The XRPD pattern of Form V is shown in FIG. 11 and the peak data is given below in Table 12.

TABLE 12

XRPD Peak Data for Form V

| 2-Theta | Height |
|---|---|
| 6.1 | 947 |
| 9.7 | 4746 |
| 11.0 | 76 |
| 12.2 | 266 |
| 12.5 | 494 |
| 13.9 | 248 |
| 14.4 | 90 |
| 15.4 | 433 |
| 16.3 | 160 |
| 17.0 | 110 |
| 18.3 | 521 |
| 18.9 | 460 |
| 19.4 | 572 |
| 21.3 | 671 |
| 22.2 | 347 |
| 23.3 | 632 |
| 23.9 | 388 |
| 28.0 | 123 |
| 28.3 | 162 |
| 29.7 | 70 |
| 36.9 | 51 |

Form V exhibits a DSC thermogram having an endotherm with an onset temperature of about 62° C. and a peak at a temperature of about 78° C.; an endotherm with an onset temperature of about 88° C. and a peak at a temperature of about 96° C.; and an endotherm with an onset temperature at about 189° C. and a peak at a temperature of about 192° C. Form V shows a weight loss of about 0.4% when heated from 50° C. to 110° C. FIG. 12 shows a DSC thermogram and a TGA thermogram of Compound 1 Form V. The data suggest that Form V may be a hydrate.

### Example 12. Characterization of Form VI

The XRPD pattern of Form VI is shown in FIG. 13 and the peak data is given below in Table 13.

TABLE 13

XRPD Peak Data for Form VI

| 2-Theta | Height |
|---|---|
| 6.3 | 154 |
| 8.9 | 483 |
| 9.6 | 117 |
| 11.0 | 105 |
| 11.9 | 247 |
| 12.8 | 65 |
| 15.7 | 194 |
| 16.3 | 109 |
| 16.6 | 425 |
| 18.0 | 577 |
| 18.4 | 280 |
| 19.0 | 2231 |
| 20.3 | 635 |
| 21.1 | 2122 |
| 22.3 | 609 |
| 22.9 | 941 |
| 24.1 | 553 |
| 24.7 | 159 |
| 25.3 | 74 |
| 25.9 | 517 |
| 26.2 | 221 |
| 26.6 | 64 |
| 27.1 | 65 |
| 27.9 | 280 |
| 29.4 | 64 |
| 29.9 | 107 |
| 30.5 | 158 |
| 32.2 | 89 |
| 33.0 | 108 |
| 33.5 | 158 |
| 37.8 | 60 |
| 39.3 | 95 |

Form VI exhibits a DSC thermogram having an endotherm with an onset temperature of about 124° C. and a peak at a temperature of about 127° C.; and a broad endotherm with an onset temperature at about 186° C. Form VI shows a weight loss of about 13.4% when heated to 190° C. FIG. 14 shows a DSC thermogram and a TGA thermogram of Compound 1 Form VI. The data suggest that Form V may be a DMSO solvate.

### Example 13. Thermal Treatment Study

Thermal treatment of Form II was conducted using DSC with the parameters below.
Equilibrate 25.0° C.
   Ramp 10° C./min to 200.0° C.
Isothermal 2.0 min
   Ramp 10° C./min to -20.0° C.
Isothermal 2.0 min
   Ramp 10° C./min to 250.0° C.

No new crystal forms were obtained by thermal treatment of Form II. Glass transition with T_{g} of 64° C. was observed in the second heating cycle.

### Example 14. Mechanical Treatment Study

Form II was ground by pestle and mortar for 2 mins and 5 mins and then analyzed by XRPD. The XRPD pattern of Form II remained unchanged after grinding, and the crystallinity of Form II decreased slightly.

### Example 15. Water Activity Study

The mixture of Form I, Form II and Form IV was suspended in a mixed solvent of water/EtOH or water/IPAc with different water content (pre-saturated, loading conc. 100 mg/mL) and stirred at 25° C. and 50° C., respectively. The remaining solid was analyzed by XRPD after 24 h. Results of the study are shown in Table 14 below.

TABLE 14

Results of Water Activity Study

| No. | Temp.(°C) | V_{Water} %* | Water activity/a_{w}** | Result |
|---|---|---|---|---|
| 1 | 25 | 0.03% | 0.0025 | Form I + Form II (15 days) |
| 2 | | 3% | 0.22 | Form I + Form II (15 days) |
| 3 | | 12% | 0.54 | Form II |
| 4 | | 32% | 0.77 | Form IV |
| 5 | | 74% | 0.92 | Form IV |
| 6 | 50 | 0.03% | 0.0025 | Form II |
| 7 | | 3% | 0.22 | Form II |
| 8 | | 12% | 0.54 | Form II |
| 9 | | 32% | 0.77 | Form II |
| 10 | | 74% | 0.92 | Form II (3 days) |
| 11 | 25 | 0.016% | 0.023 | Form II |
| 12 | | 0.22% | 0.22 | Form II |

^{∗}V_{water}% is the percent volume of water used in this study.
^{∗∗}aw is the water activity, or partial vapor pressure of water in a solution divided by the standard state partial vapor pressure of water.

### Example 16. Crystal Form Stability Study

Certain amounts of Form II were added into sesame oil to make suspensions with different loading concentrations (10 mg/mL and 100 mg/mL). The suspensions were kept stirring at RT for 7 days. Solid samples were collected by filtration and analyzed by XRPD to check the crystal form.

The results (shown in Table 15 below) indicated that the XRPD pattern of Form II was unchanged in sesame oil for 7 days.

TABLE 15

Stability in Sesame Oil

| Media | Temp. | Loading Conc. (mg/mL) | Results |
|---|---|---|---|
| Sesame Oil | RT | 10 | Form II |
| | | 100 | Form II |

### Example 17. Solid-State Stability Study

Appropriate amount of Form II were placed at 40° C./75%RH (open) and 60° C./capped for up to 14 days, respectively. At day 0, 7 and 14, the sample was dissolved in diluent to prepare solution for HPLC purity analysis. Solid samples were analyzed by XRPD to check the crystal form.

Physical and chemical stability of Form II was conducted at 60° C. (closed) and 40° C./75%RH (open) up to 14 days, samples were prepared in duplicate for each condition. The result indicated that Form II was both physically and chemically stable at 60° C. (closed) and 40° C./75%RH for 14 days. The results are summarized in Table 16 below.

TABLE 16

Stability Evaluation Results

| Sample | Initial Purity (Area%) | Purity-7 d (Area%) /XRPD | | Purity-14 d (Area%) /XRPD | |
|---|---|---|---|---|---|
| | | 40° C./75%RH | 60° C. | 40° C./75%RH | 60° C. |
| Form II | 99.37 | 99.37/99.35/ unchanged | 99.38/99.35/ unchanged | 99.40/99.38/ unchanged | 99.37/99.33/ unchanged |

### Example 18. Preparation of Form II at 300 Mg Scale

About 300 mg of Compound 1 Form I (as prepared in Example 1) was suspended in 2.0 mL ethyl acetate. The suspension was kept stirring at RT (~25° C.) for 4 days. Solid was collected by filtration and dried at 40° C. in vacuum for 3 h, and 270 mg of Form II was obtained in 90% yield andcharacterized by XRPD, PLM, TGA and DSC. PLM image showed that Form II was irregular shaped crystals with particle size less than 10 µm. An endothermic peak with onset temperature of 190° C. was detected by DSC and there was no obvious weight loss before 150° C. in the TGA profile. DVS data showed 0.38% weight change from 0.0%RH to 80%RH andwas slightly hygroscopic. The crystal form did not change after DVS testing.

### Example 19. Solubility of Form II in Common Solvents

An appropriate amount of Form II was weighed into a sample vial and then the indicated solvent was added gradually until all the solid was dissolved at RT. The dissolution was observed visually. The results are presented in Table 17.

TABLE 17

Dynamic solubility data of Form II in single solvents

| No. | Solvent | Abbreviation | Solubility (22° C., mg/mL) |
|---|---|---|---|
| 1 | Acetic acid | / | > 167 |
| 2 | Dichloromethane | DCM | 16.7 |
| 3 | Dimethyl sulfoxide | DMSO | 12.5 |
| 4 | Methanol | MeOH | 8.3 |
| 5 | Tetrahydrofuran | THF | 4.2 |
| 6 | Methyl ethyl ketone | MEK | 1.7 |
| 7 | Ethanol | EtOH | 1.0 |
| 8 | Acetone | / | 0.8 |
| 9 | 2-Me-THF | 2-Me-THF | 0.7 |
| 10 | Acetonitrile | ACN | 0.6 |
| 11 | Tert-Butyl methyl ether | MTBE | <0.6 |
| 12 | Ethyl acetate | EA | <0.6 |
| 13 | Isopropyl acetate | IPAc | <0.6 |
| 14 | n-Heptane | Hept | <0.6 |
| 15 | 2-Propanol | IPA | <0.6 |
| 16 | Toluene | Tol | <0.6 |
| 17 | Water | / | <0.6 |

### Example 20. Solubility of Form II in Mixed Solvents

The solubility of Form II was assessed in various solvent mixtures. An excess amount of Form II was weighted into 1 mL of selected solvents to form a suspension which was stirred at 20° C. under 800 rpm for 30 minutes, then heated to 65° C. at 0.1° C./min. The results are shown in Tables 18-21 below.

TABLE 18

Dynamic solubility data of Form II in Mixed Solvents at RT

| No. | Solvent | Volume Ratio | Solubility (mg/mL) |
|---|---|---|---|
| 1 | MeOH/Anisole | 1/1 | 150-160 |
| 2 | MeOH/water | 9/1 | 35-43 |
| 3 | MeOH/MEK | 1/1 | 20-30 |
| 4 | MeOH/MEK | ½ | 24-30 |
| 5 | MeOH/Ethyl formate | ½ | <25 |
| 6 | MeOH/Acetone | ½ | <14 |
| 7 | MeOH/EA | ½ | <10 |
| 8 | Ethyl formate/diethyl ether | ½ | 7-8 |
| 9 | MeOH/MEK | ⅑ | 7-8 |
| 10 | Ethyl formate/n-BuOH | ½ | 3-4 |
| 11 | MeOH/n-BuOH | ½ | <3 |
| 12 | MeOH/Diethyl ether | ½ | <2 |
| 13 | Ethyl formate/Acetone | ½ | <2 |
| 14 | Ethyl formate/MEK | ½ | <2 |
| 15 | Ethyl formate/MTBE | ½ | <2 |
| 16 | Ethyl formate/IPA | ½ | <2 |
| 17 | MEK/Ethyl formate | ½ | <1 |
| 18 | MeOH/Ethyl formate | ⅑ | <1 |
| 19 | MEK/n-BuOH | ½ | <1 |
| 20 | Ethyl formate/EA | ½ | <1 |
| 21 | Ethyl formate/IPAC | ½ | <1 |
| 22 | MEK/diethyl ether | ½ | <1 |
| 23 | MEK/IPAC | ½ | <1 |
| 24 | MEK/EA | ½ | <1 |
| 25 | MEK/IPA | ½ | <1 |
| 26 | MEK/Diethyl ether | ½ | <1 |

Dissolution was observed visually.

TABLE 19

Dynamic Solubility data of Form II in MeOH/Water

| No. | Solvent | 0° C. | RT | 50° C. | 60° C. |
|---|---|---|---|---|---|
| 1 | MeOH | ND | 8.3 | 29-33 | 71-83 |
| 2 | MeOH/water (9/1) | 6-10 | 10-13 | ND | >125 |
| 3 | MeOH/water (8/2) | ND | 13-17 | 50-100 | >125 |
| 4 | MeOH/water (7/3) | ND | 10-13 | ND | ~125 |
| 5 | MeOH/water/MTBE (9/1/10) | >4.5 | ND | ND | ND |
| 6 | MeOH/water/MTBE (9/1/20) | ~3 | ND | ND | ND |

Dissolution was observed visually. Solubility data was in mg/mL. ND = not determined.

TABLE 20

Kinetic solubility data of Form II in MeOH/Water

| No. | Solvent | Volume Ratio | Form II / mg | T_{dissolve} / °C |
|---|---|---|---|---|
| 1 | MeOH/Water | 9/1 | 150.24 | 61.9 |
| 2 | | | 125.35 | 60.3 |
| 3 | | | 100.46 | 56.7 |
| 4 | | | 81.37 | 52.5 |
| 5 | | | 61.14 | 47.1 |
| 6 | | | 40.30 | 40.4 |

Clear temperature point was detected by Crystal 16 equipment.

TABLE 21

Kinetic solubility data of Form II in MeOH-Water/MTBE

| No. | Solvent | Volume Ratio | Form II / mg | T_{dissolve} / °C |
|---|---|---|---|---|
| 1 | MeOH-Water (9/1, v/v)/MTBE | 10/0 | 151.90 | 63.7 |
| 2 | | 9/1 | 101.46 | 58.0 |
| 3 | | 8/2 | 70.64 | 53.1 |
| 4 | | 7/3 | 62.04 | 51.4 |
| 5 | | 6/4 | 51.90 | 54.1 |
| 6 | | 1/1 | 21.14 | 40.6 |
| 7 | | ½ | 11.58 | 41.6 |
| 8 | | ⅓ | 6.51 | 30.3 |

Clear temperature point was detected by Crystal 16 equipment. Water acts as co-solvent to improve solubility. MTBE was not a potent antisolvent as the solubility was 6.5 mg/mL at 30° C. when the volume ratio of MeOH-water/MTBE was 9/1/30.

Tables 22-24 show the solubility of Form II in MeOH-Anisole/MTBE (or heptane). The solubility data was used to control supersaturation and calculate theoretical yield of the crystallization process.

TABLE 22

Dynamic solubility data of Form II in MeOH/ Anisole

| No. | Solvent | 25° C., mg/mL | 40° C., mg/mL |
|---|---|---|---|
| 1 | Anisole | <1 | ND |
| 2 | MeOH | 8.3 | ND |
| 3 | MeOH/Anisole (½) | ~150 | >200 |
| 4 | MeOH/Anisole (1/1) | 163-176 | ~250 |
| 5 | MeOH/Anisole (2/1) | 96-106 | ND |
| 6 | MeOH/Anisole/Hept (1/2/3) | >10 | ND |
| 7 | MeOH/Anisole/Hept (1/2/6) | ~5 | ND |
| 8 | MeOH/Anisole/MTBE (1/2/6) | ~3 | ND |
| 9 | MeOH/Anisole/Hept (1/1/6) | <5 | ND |

Dissolution was observed visually. ND = not determined.

TABLE 23

Kinetic solubility data of Form II in MeOH/Anisole

| No. | Solvent | Volume Ratio | Form II / mg | T_{dissolve} / °C |
|---|---|---|---|---|
| 1 | MeOH/Anisole | 1/1 | 250.89 | 40.3 |
| 2 | | | 230.40 | 37.1 |
| 3 | | | 210.75 | 34.1 |
| 4 | | | 190.72 | 33.9 |
| 5 | | | 171.92 | 27.0 |

Clear temperature point was detected by Crystal 16 equipment.

TABLE 24

Kinetic solubility data of Form II in MeOH-Anisole/MTBE

| No. | Solvent | Volume Ratio | Form II / mg | T_{dissolve} / °C |
|---|---|---|---|---|
| 1 | MeOH-Anisole (1/1, v/v)/MTBE | 9/1 | 190.37 | 40.8 |
| 2 | | 8/2 | 131.08 | 42.8 |
| 3 | | 7/3 | 80.36 | 47.3 |
| 4 | | 6/4 | 51.0 | 46.3 |
| 5 | | 1/1 | 25.06 | 42.4 |
| 6 | | ½ | 7.12 | 33.6 |
| 7 | | ⅓ | 4.83 | 50.2 |

Clear temperature point was detected by Crystal 16 equipment.

### Example 21. Anti-Solvent Crystallization

Anti-solvent crystallization was conducted to screen crystallization solvents of Form II. About 50 mg of Compound 1 Form I was dissolved in the selected solvents. A supersaturated solution at RT was created by adding anti-solvent or dissolving the solid at room temperature. Form II crystal seeds were added and stirred for 10 minutes, then the anti-solvent was added dropwise until precipitation occurred. The results are shown in Table 25 below.

TABLE 25

Results of anti-solvent crystallization

| Solvent, v/v | Anti-solvent, v/v | Solvent/ Anti-solvent, v/v | Result | |
|---|---|---|---|---|
| | | | 30 min | 20 h |
| Acetic acid | IPA/Hept (1/1) | ¼ | Pattern D | Pattern D |
| MeOH/Anisole(½) | Hept | ½ | Oil | Form II |
| MeOH/Anisole(½) | MTBE | ½ | Form II | Form II |

Samples of Pattern D were not further isolated or characterized. Pattern D is likely an acetic acid solvate.

### Example 22. Competitive Slurry of Form I and Form II

To study the effect of solvent composition on crystal forms, a competitive slurry experiment was carried out between Form I and Form II in potential solvent systems. The same amount of Form I and Form II (15 mg) were mixed and added in 1 mL of MeOH/water/MTBE (9/1/20, v/v/v) or MeOH/Anisole/MTBE (1/1/6, v/v/v) and then slurried at RT or 0° C. for 20 h and the results are summarized in Table 26. Form I is more stable than Form II in MeOH/Water (9/1, v/v) at 0° C. and 10° C. In MeOH/water/MTBE (9/1/20, v/v/v) and MeOH/Anisole/MTBE (1/1/6, v/v/v), Form II is more stable.

TABLE 26

Results of competitive slurry

| | | | |
|---|---|---|---|
| Input | Solvent | Temp. (°C) | Output |
| Form I +Form II (15 mg each) | MeOH/Water (9/1, v/v) | 0 | Form I |
| | | 10 | |
| | | RT | Form II |
| Form I +Form II (15 mg each) | MeOH/water/MTBE (9/1/20, v/v/v) | RT | Form II |
| Form I +Form II (15 mg each) | MeOH/Anisole/MTBE (1/1/6, v/v/v) | RT | Form II |

### Example 23. Crystallization of Form II in MeOH-Water/MTBE

As Form I is a hydrate, addition of water raises the risk of formation of Form I. According to the above data, Form II is more stable than Form I in MeOH-Water (9/1,v/v)/MTBE (½, v/v) at RT. Form I is more stable at low temperatures in the methanol-water system. Crystallization of Form II was carried out in MeOH-Water/MTBE solvent system. XRPD and thermograms confirmed Form II was successfully prepared in 75% yield. ¹H-NMR showed 0.1%w/w of MeOH and 0.1%w/w of MTBE, which were within the concentration limit.

### Example 24. Crystallization of Form II in MeOH-Anisole/MTBE

To improve the solubility of Form II in MeOH, anisole was added as a co-solvent.

Compound 1 was dissolved in anisole/methanol 1:1 v/v (2 rel. volumes in total) at approximately 55° C. The resulting solution was polish filtered to remove insoluble matter, after which MTBE (6 rel. volumes) was added at 55° C. over a period of not less than an hour. Then, Compound 1 seed material of the correct polymorphic form was charged. MTBE (6 rel. volumes) was again added at 55° C. over a period of at least an hour. The mixture was stirred at approx. 55° C. for at least an hour, cooled down to approx. 25° C., stirred for at least an hour, and filtered. The filter cake was washed with MTBE (2 rel. volumes) and dried to afford Form II.

According to the above experimental data, the ternary solvent system (MeOH-Anisole/MTBE) is suitable for crystallization of Form II.

### Example 25. Micronization of Form II

Micronization was performed by a Micron Jet Mill Pilot. The detailed parameters are listed as below.
Instrument: Micron Jet Mill Pilot (NB-MS-JEM-1)
Feeding speed: Manual addition according to the practical size test result
Feeding pressure: 0.35-0.40 MPa
Milling Pressure: 0.30-0.35 MPa
Screw speed: 20 rpm

6.7 g of Form II was subjected to micronization. 5.4 g of milled sample was obtained with a yield of 80%. The crystal form remained unchanged after micronization. The particle size of milled sample was less than 5 µm under PLM. PSD results indicated that the particle size milled down from D(90) ~98 µm to D(90) ~3.34 µm. The detailed result of PSD is listed in Table 27.

TABLE 27

PSD Result of the Initial and Milled Sample

| D(10)µm | D(50)µm | D(90)µm |
|---|---|---|
| 7.97 | 39.60 | 98.0 |
| 0.77 | 1.71 | 3.34 |

### Example 26. Thermal Treatment of Form I and II

To study the temperature effect on crystal form, thermal treatment of Form I and Form II mixture was performed. About 100 mg of a Form II and Form I mixture was heated to 100° C. for up to 20 h. Solid was collected at 3 h and 20 h for HPLC and XRPD analysis. Form I converted to Form II after drying at 100° C. for 3 h and the purity remained unchanged even after 20 h.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A solid form of Compound 1 having the formula:. 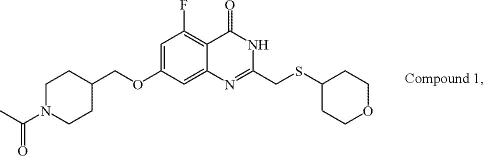 [SMILES: CC(=O)N1CCC(COc2cc(F)c3c(=O)[nH]c(CSC4CCOCC4)nc3c2)CC1]
wherein the solid form is crystalline.

2. The solid form of claim 1, which is anhydrous.

3. The solid form of claim 2, which is Form II.

4. The solid form of claim 3, having at least one characteristic XRPD peak selected from about 8.6, about 9.4, about 12.2, about 14.8, about 15.9, about 16.1, about 17.7, about 18.1, about 19.5, about 20.0, about 22.7, and about 24.6 degrees 2-theta.

5. The solid form of claim 3, having at least two characteristic XRPD peaks selected from about 8.6, about 9.4, about 12.2, about 14.8, about 15.9, about 16.1, about 17.7, about 18.1, about 19.5, about 20.0, about 22.7, and about 24.6 degrees 2-theta.

6. The solid form of claim 3, having an XRPD pattern with characteristic peaks as substantially shown in FIG. 4.

7. The solid form of claim 3, having a DSC thermogram comprising an endotherm peak at a temperature of about 190° C.

8. The solid form of claim 3, having a DSC thermogram substantially as depicted in FIG. 5.

9. The solid form of claim 1, which is a hydrate.

10. The solid form of claim 9, which is Form I.

11. The solid form of claim 10, having at least one characteristic XRPD peak selected from about 6.3, about 8.9, about 12.5, about 14.3, about 15.3, about 16.9, about 17.7, about 19.0, about 22.4, and about 23.8 degrees 2-theta.

12. The solid form of claim 10, having at least two characteristic XRPD peaks selected from about 6.3, about 8.9, about 12.5, about 14.3, about 15.3, about 16.9, about 17.7, about 19.0, about 22.4, and about 23.8 degrees 2-theta.

13. The solid form of claim 10, having an XRPD pattern with characteristic peaks as substantially shown in FIG. 1.

14. The solid form of claim 10, having a DSC thermogram comprising an endotherm peak at a temperature of about 192° C.

15. The solid form of claim 10, having a DSC thermogram substantially as depicted in FIG. 2.

16. The solid form of claim 9, which is Form III.

17. The solid form of claim 16, having at least one characteristic XRPD peak selected from about 6.2, about 8.9, about 12.3, about 14.2, about 15.5, about 16.9, about 18.1, about 19.0, about 21.5, about 21.9, and about 23.8 degrees 2-theta.

18. The solid form of claim 16, having at least two characteristic XRPD peaks selected from about 6.2, about 8.9, about 12.3, about 14.2, about 15.5, about 16.9, about 18.1, about 19.0, about 21.5, about 21.9, and about 23.8 degrees 2-theta.

19. The solid form of claim 16, having an XRPD pattern with characteristic peaks as substantially shown in FIG. 8.

20. The solid form of claim 9, which is a dihydrate.

21. The solid form of claim 20, which is Form IV.

22. The solid form of claim 21, having at least one characteristic XRPD peak selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta.

23. The solid form of claim 21, having at least two characteristic XRPD peaks selected from about 6.3, about 9.2, about 10.7, about 13.4, about 15.0, about 16.7, about 18.5, about 18.7, about 20.0, about 20.4, about 22.6, about 22.8, about 23.2, and about 23.7 degrees 2-theta.

24. The solid form of claim 21, having an XRPD pattern with characteristic peaks as substantially shown in FIG. 9.

25. The solid form of claim 21, having a DSC thermogram comprising an endotherm peak at a temperature of about 192° C.

26. The solid form of claim 21, having a DSC thermogram substantially as depicted in FIG. 10.

27. The solid form of claim 9, which is Form V.

28. The solid form of claim 27, having at least one characteristic XRPD peak selected from about 6.1, about 9.7, about 12.2, about 12.5, about 15.4, about 18.3, about 18.9, about 19.4, about 21.3, about 22.2, and about 23.3 degrees 2-theta.

29. The solid form of claim 27, having at least two characteristic XRPD peaks selected from about 6.1, about 9.7, about 12.2, about 12.5, about 15.4, about 18.3, about 18.9, about 19.4, about 21.3, about 22.2, and about 23.3 degrees 2-theta.

30. The solid form of claim 27, having an XRPD pattern with characteristic peaks as substantially shown in FIG. 11.

31. The solid form of claim 27, having a DSC thermogram comprising an endotherm peak at a temperature of about 192° C.

32. The solid form of claim 27, having a DSC thermogram substantially as depicted in FIG. 12.

33. The solid form of claim 1, which is a DMSO solvate.

34. The solid form of claim 33, which is Form VI.

35. The solid form of claim 34, having at least one characteristic XRPD peak selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta.

36. The solid form of claim 34, having at least two characteristic XRPD peaks selected from about 6.3, about 8.9, about 11.9, about 15.7, about 16.6, about 18.0, about 19.0, about 20.3, about 21.1, about 22.3, about 22.9, about 24.1, about 25.9, and about 27.9 degrees 2-theta.

37. The solid form of claim 34, having an XRPD pattern with characteristic peaks as substantially shown in FIG. 13.

38. The solid form of claim 34, having a DSC thermogram comprising an endotherm peak at a temperature of about 127° C.

39. The solid form of claim 34, having a DSC thermogram substantially as depicted in FIG. 14.

40. A pharmaceutical composition comprising a solid form of claim 1 and at least one pharmaceutically acceptable carrier.

41. A method of inhibiting the activity of PARP14 comprising contacting a solid form of claim 1 with said PARP14.

42. A method of decreasing IL-10 in a cell comprising contacting a solid form of claim 1 with said cell.

43. A method of treating cancer in a patient in need of treatment comprising administering to said patient a therapeutically effective amount of a solid form of claim 1.

44. The method of claim 43 wherein said cancer is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

45. A method of treating an inflammatory disease in a patient in need of treatment comprising administering to said patient a therapeutically effective amount of a solid form of claim 1.

46. The method of claim 45, wherein the inflammatory disease is selected from asthma, atopic dermatitis, psoriasis, rhinitis, systemic sclerosis, keloids, an eosinophilic disorder, pulmonary fibrosis, and a type 2 cytokine pathology.
